(19) United States
(12) Patent Application Publication
(10) Pub. No.: US 2015/0037893 A1
(43) Pub. Date: Feb. 5, 2015

(54) PATHOGEN RESISTANT PLANT CELLS AND METHODS OF MAKING

(71) Applicants: CARNEGIE INSTITUTION OF WASHINGTON, Washington, DC (US); THE GENERAL HOSPITAL CORPORATION, Boston, MA (US)

(72) Inventors: Frederick M. Ausubel, Boston, MA (US); Cristian H. Danna, Boston, MA (US); Wolf B. Frommer, Washington, DC (US)

(21) Appl. No.: 14/387,450

(22) PCT Filed: Mar. 25, 2013
(86) PCT No.: PCT/US13/33677

§ 371 Date: Sep. 23, 2014

Related U.S. Application Data
(60) Provisional application No. 61/615,216, filed on Mar. 24, 2012.
(60) Provisional application No. 61/615,512, filed on Mar. 26, 2012.

(51) Int. Cl. C12N 15/82

(52) U.S. Cl. CPC C12N 15/8279; C12N 15/8201
USPC 435/468; 435/419

(57) ABSTRACT

The present invention relates to genetically modified plant cells that have reduced expression or activity of at least one amino acid efflux transporter and/or at least one mineral efflux transporter compared to levels of expression or activity of the at least one amino acid efflux transporter or mineral efflux transporter in an unmodified plant cell. The present invention also relates to genetically modified plant cells that have increased expression or activity of at least one amino acid influx transporter and/or at least one mineral influx transporter compared to levels of expression or activity of the at least one amino acid influx transporter or mineral influx transporter in an unmodified plant cell.

### STATEMENT REGARDING FEDERALLY SPONSORED RESEARCH OR DEVELOPMENT

Part of the work performed during development of this invention utilized U.S. Government funds from National Institutes of Health Grant No. R37 GM48707 and National Science Foundation Grant No. MCB-0519898. The U.S. Government has certain rights in this invention.

### BACKGROUND OF THE INVENTION

1. Field of the Invention

The present invention relates to genetically modified plant cells that have altered expression or activity of at least one amino acid efflux transporter and/or at least one mineral efflux transporter compared to levels of expression or activity of the at least one amino acid efflux transporter or mineral efflux transporter in an unmodified plant cell. The present invention also relates to genetically modified plant cells that have altered expression or activity of at least one amino acid influx transporter and/or at least one mineral influx transporter compared to levels of expression or activity of the at least one amino acid influx transporter or mineral influx transporter in an unmodified plant cell.

2. Background of the Invention

Pro- and eukaryotes all depend on adequate supply with nutrients. These nutrients can be inorganic or organic, such as sugars, amino acids, metal ions, minerals, and vitamins and include all of the macro and micronutrients. Quantitatively, carbon-containing compounds such as sucrose, glucose, other mono- and disaccharides, oligosaccharides such as raffinose, starch and alcohols such as mannitol, sorbitol or glycerol, serve to supply carbon and energy. Other nutrients include organic acids such as malate or citrate. The second most abundant nutrients are N-containing compounds such as amino acids, which also supply carbon and nitrogen for DNA, RNA and protein synthesis. Additional nutrients include but are not limited to sulfur and phosphorus, water, calcium, magnesium, iron, zinc, copper, cadmium, manganese, molybdenum, and vitamins, i.e. all compounds that the organism cannot synthesize from scratch.

Plants transport nutrients to provide essential elements to the varying tissues which may not necessarily receive them otherwise. For example, roots systems are able to receive nutrients and transporters assist to transport them through to other tissues within the plant. Similarly, some tissues transform or assemble nutrients and export them to other tissues. Nutrients may include sugars, vitamins, water, amino acids and nucleotides and folic acid. For example, plants transport fixed carbon predominantly as sucrose, which is produced in mesophyll cells by photosynthesis and imported into phloem cells for translocation throughout the plant.

Plant pathogens are no different than other organisms in their requirements for nutrients such as amino acids, and they require the ability to obtain nutrients from host plants to grow and propagate. Indeed, plant pathogens have developed mechanisms to utilize the transporters of their host plants to their benefit and the detriment of the host. For example, it is possible that pathogens may suppress activation influx transporter proteins to make nutrients available at the places where they grow, such as the intercellular space, the vasculature, etc. Thus, limiting a plant pathogen's access to amino acids may reduce the pathogen's ability to grow and propagate. Accordingly, pathogen resistance or tolerance may be conferred in plants in which there is a reduced capacity for the pathogen to obtain amino acids or other nutrients from plant cells.

### SUMMARY OF THE INVENTION

The present invention relates to genetically modified plant cells that have increased or decreased expression or activity of at least one amino acid efflux transporter compared to levels of expression or activity of the at least one amino acid efflux transporter in an unmodified plant cell.

The present invention also relates to methods of producing pathogen-resistant plant cells, with the methods comprising identifying at least one amino acid efflux transporter wherein the levels of expression or activity of the at least one amino acid efflux transporter are altered in the plant cell in response to an infection of the pathogen as compared to an uninfected plant cell, and subsequently modifying the plant cell to either increase or decrease the activity or the expression of the at least one identified amino acid efflux transporter, whereby increasing or decreasing the activity or the expression of the at least one identified amino acid efflux transporter produces the pathogen-resistant plant cell.

The present invention relates to genetically modified plant cells that have increased or decreased expression or activity of at least one mineral efflux transporter compared to levels of expression or activity of the at least mineral efflux transporter in an unmodified plant cell.

The present invention also relates to methods of producing pathogen-resistant plant cells, with the methods comprising identifying at least one mineral efflux transporter wherein the levels of expression or activity of the at least one mineral efflux transporter are altered in the plant cell in response to an infection of the pathogen as compared to an uninfected plant cell, and subsequently modifying the plant cell to increase or decrease the activity or the expression of the at least one identified mineral efflux transporter, whereby increasing or decreasing the activity or the expression of the at least one identified mineral efflux transporter produces the pathogen-resistant plant cell.

The present invention relates to genetically modified plant cells that have increased or decreased expression or activity of at least one amino acid influx transporter compared to levels of expression or activity of the at least one amino acid influx transporter in an unmodified plant cell.

The present invention also relates to methods of producing pathogen-resistant plant cells, with the methods comprising identifying at least one amino acid influx transporter wherein the levels of expression or activity of the at least one amino acid transporter are altered in the plant cell in response to an infection of the pathogen as compared to an uninfected plant cell, and subsequently modifying the plant cell to increase or decrease the activity or expression of the at least one identified amino acid influx transporter, whereby increasing or decreasing the activity or expression of the at least one identified amino acid influx transporter produces the pathogen-resistant plant cell.

The present invention relates to genetically modified plant cells that have increased or decreased expression or activity of at least one mineral influx transporter compared to levels of expression or activity of the at least mineral influx transporter in an unmodified plant cell.

The present invention also relates to methods of producing pathogen-resistant plant cells, with the methods comprising identifying at least one mineral influx transporter wherein the levels of expression or activity of the at least one mineral influx transporter are altered in the plant cell in response to an infection of the pathogen as compared to an uninfected plant cell, and subsequently modifying the plant cell to increase or decrease the activity or expression of the at least one identified mineral influx transporter, whereby increasing or decreasing the activity or expression of the at least one identified mineral influx transporter produces the pathogen-resistant plant cell.

### BRIEF DESCRIPTION OF THE DRAWINGS

FIG. 1 depicts the growth of P. syringae-LUX in Arabidopsis seedlings that were untreated (C) or treated with Flg22 (F). Growth of was measured in the seedlings or in the seedling growth medium (exudates) after 2 hours or 24 hours.

FIG. 2 depicts levels of reducing sugars that were measured in the exudates when Arabidopsis seedlings were grown in medium and were untreated (C) or treated with Flg22 (F). Levels of sugar were measured using the Somogyi Nelson method. Arabidopsis exudates were lyophilized and reconstituted with water to 3 mg/ml.

FIG. 3 depicts arbitrary levels of reducing sugars in exudates following growth of P. syringae. The two measurements at the left are controls in the absence of bacterial growth. On the right, two different P. syringae strains, Psm and Pst, were grown in exudates from control plants (C) or plants treated with Flg22 (F). “HAI” refers to hours after infection.

FIG. 4 depicts that the addition of glucose does not suppress the ability of Flg22 to restrict the growth of P. syringae. Seedlings were either untreated (C) or treated with Flg22 (F) and then inoculated with Pst with or without supplementation with glucose.

FIG. 5 depicts that several amino acids were present in reduced amounts in seedling exudates following treatment with Flg22. The exudates were analyzed by HPLC and individual amino acid peaks were identified in comparison with the retention times of individual amino acids. “flgt” refers to Flg22-treated seedlings.

FIG. 6 depicts that glutamate supplementation allows P. syringae (Pst) to grow in Murashige and Skoog Basal medium used to grow the Arabidopsis seedlings.

FIG. 7 depicts that amino acid supplementation allows growth of Pst or Psm in exudates from Flg22-treated seedlings.

FIG. 8 depicts that supplementation with various amino acids suppresses the growth-limiting effect of Flg22 treatment.

FIG. 9 depicts bacterial growth inhibition triggered by flg22 elicitation of seedlings. Black bars correspond to bacteria growth in mock treated seedlings conditions. Clear bars correspond to bacterial growth in flg22-elicited seedlings. Crossed bars pattern indicate statistically significant differences compared to bacterial growth in wild type seedlings treated with flg22. Three independent experiments showed similar results

FIG. 10 depicts the ability of coronatine to modify the availability of amino acids in the intercellular fluids. The concentration of glutamine was higher in the presence of Coronatine and the combination of fagellin and coronatine only brought glutamine concentrations back to control levels, suggesting that coronatine may eliminate bacterial growth inhibition triggered by flg22. When the seedlings were co-treated with flagellin and coronatine the bacterial growth inhibition was no longer observed.

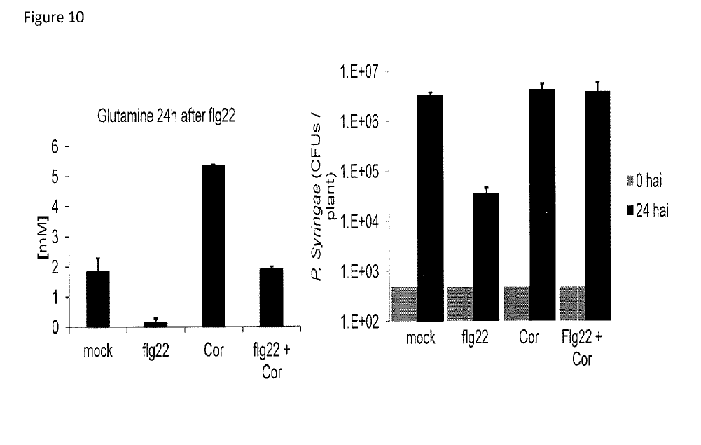

### DETAILED DESCRIPTION OF THE INVENTION

The present invention relates to genetically modified plant cells that have increased or decreased expression or activity of at least one amino acid and/or mineral efflux transporter compared to levels of expression or activity of the at least one amino acid and/or mineral efflux transporter in an unmodified plant cell. The present invention also relates to genetically modified plant cells that have increased or decreased expression or activity of at least one amino acid and/or mineral influx transporter compared to levels of expression or activity of the at least one amino acid and/or mineral influx transporter in an unmodified plant cell.

As described herein, the genetically modified plant cell may be a plant cell from a dicot or monocot or gymnosperm. The plant may be crops, such as a food crops, feed crops or biofuels crops. Exemplary important crops may include corn, wheat, soybean, cotton and rice. Crops also include corn, wheat, barley, triticale, soybean, cotton, millet, sorghum, sugarcane, sugar beet, potato, tomato, grapevine, citrus (orange, lemon, grapefruit, etc), lettuce, alfalfa, common bean, fava bean and strawberries, sunflowers and rapeseed, cassava, miscanthus and switchgrass. Other examples of plants include but are not limited to an African daisy, African violet, alfalfa, almond, anemone, apple, apricot, asparagus, avocado, azalea, banana and plantain, beet, bellflower, black walnut, bleeding heart, butterfly flower, cacao, caneberries, canola, carnation, carrot, cassava, diseases, chickpea, cineraria, citrus, coconut palm, coffee, common bean, maize, cotton, crucifers, cucurbit, cyclamen, dahlia, date palm, douglas-fir, elm, English walnut, flax, Acanthaceae, Agavaceae, Araceae, Araliaceae, Araucariacea, Asclepiadaceae, Bignoniaceae, Bromeliaceae, Cactaceae, Commelinaceae, Euphobiaceae, Gentianaceae, Gesneriaceae, Maranthaceae, Moraceae, Palmae, Piperaceae, Polypodiaceae, Urticaceae, Vitaceae, fuchsia, geranium, grape, hazelnut, hemp, holiday cacti, hop, hydrangea, impatiens, Jerusalem cherry, kalanchoe, lettuce, lentil, lisianthus, mango, mimulus, monkey-flower, mint, mustar, oats, papaya, pea, peach and nectarine, peanut, pear, pearl millet, pecan, pepper, Persian violet, pigeonpea, pineapple, pistachio, pocketbook plant, poinsettia, potato, primula, red clover, rhododendron, rice, rose, rye, safflower, sapphire flower, spinach, strawberry, sugarcane, sunflower, sweetgum, sweet potato, sycamore, tea, tobacco, tomato, verbena, and wild rice.

The plant cell can be from any part or tissue of a plant including but not limited to the root, stem, leaf, seed, flower, fruit, anther, nectary, ovary, petal, tapetum, xylem, or phloem. If the genetically modified plant cell is comprised within a whole plant, the entire plant need not contain or express the genetic modification.

The one or more efflux transporter proteins that are modified such that their normal expression or activity is either increased or decreased can be an efflux transporter of any amino acid. For example, the amino acid transporter efflux proteins in which the expression or activity is reduced compared to levels of expression or activity of the amino acid efflux transporter in an unmodified plant cell include but are not limited to cysteine transporters, histidine transporters, isoleucine transporters, methionine transporters, serine transporters, valine transporters, alanine transporters, glycine transporters, leucine transporters, proline transporters, threonine transporters, phenylalanine transporters, arginine transporters, tyrosine transporters, tryptophan transporters, aspartate transporters, asparagine transporters, glutamate transporters, glutamine transporter and lysine transporters.

The one or more efflux transporter proteins that are modified such that their normal expression or activity is either increased or decreased can be an efflux transporter of any mineral. For example, the mineral transporter efflux proteins in which the expression or activity is reduced compared to levels of expression or activity of the mineral efflux transporter in an unmodified plant cell include but are not limited a zinc transporter, a cadmium transporter, an iron transporter, a nitrate transporter and the like.

The one or more influx transporter proteins that are modified such that their normal expression or activity is either increased or decreased can be an influx transporter of any amino acid. For example, the amino acid influx transporter proteins in which the expression or activity is increased compared to levels of expression or activity of the amino acid influx transporter in an unmodified plant cell include but are not limited to cysteine transporters, histidine transporters, isoleucine transporters, methionine transporters, serine transporters, valine transporters, alanine transporters, glycine transporters, leucine transporters, proline transporters, threonine transporters, phenylalanine transporters, arginine transporters, tyrosine transporters, tryptophan transporters, aspartate transporters, asparagine transporters, glutamate transporters, glutamine transporter and lysine transporters.

The one or more mineral transporter influx proteins that are modified such that their normal expression or activity is either increased or decreased can be an influx transporter of any mineral. For example, the mineral transporter inlux proteins in which the expression or activity is increased compared to levels of expression or activity of the mineral influx transporter in an unmodified plant cell include but are not limited a zinc transporter, a cadmium transporter, an iron transporter, a nitrate transporter and the like.

Examples of genes in Arabidopsis that encode efflux or influx transporters include but are not limited to AT5G01240, AT2G21050, AT2G38120, AT1G31820, AT1G77960, AT1G31830, AT5G49630, AT1G71680, AT5G63850, AT1G77380, AT5G40780, AT4G35180, AT3G55740, AT1G48640, AT1G08230, AT5G65990, AT5G36940, AT1G80510, AT2G34960, AT4G21120, AT1G75500, AT3G18200, AT4G08300, AT4G01440, AT1G01070, AT3G30340 and AT1G44800. Although the gene nomenclature above refers to genes identified in The Arabidopsis Information Resource (TAIR) database, which is available on the worldwide web at www.arabidopsis.org, it is understood that the invention is not limited to genes in Arabidposis and that the invention encompasses orthologs of genes in other species. For example, it is understood that methods and plant cells utilizing the transporter encoded by the gene AT4G01440 in Arabidopsis can be applied to the orthologous gene in another species. As used herein, orthologous genes are genes from different species that perform the same or similar function and are believed to descend from a common ancestral gene. Often, proteins encoded by orthologous genes have similar or nearly identical amino acid sequence identities to one another, and the orthologous genes themselves have similar nucleotide sequences, particularly when the redundancy of the genetic code is taken into account. Thus, by way of example, the ortholog of an efflux histidine transporter in Arabidopsis would be an efflux histidine efflux transporter in another species of plant, regardless of the amino acid sequence of the two proteins.

As used herein, pathogen refers to an organism that utilizes plant nutrients to grow and divide. Pathogens may include pests and parasites, e.g., mycoparasites, mycoplasma-like organism (MLO), a Rickettsia-Like Organism (RLO), bacteria, or molds. The pathogen to which the plant cell is modified to become resistant or tolerant includes but is not limited to bacteria or fungi. Pathogens also include organisms that cause infectious diseases, such as but not limited to fungi, oomycetes, bacteria, protozoa, nematodes and parasitic plants.

As used herein, a plant cell that is pathogen resistant is a plant cell that will not support the growth and/or propagation of a pathogen such that a pathogen will not survive in the plant cell or in the environment or vicinity immediately surrounding the genetically modified plant cell. A plant cell that is pathogen tolerant is a plant cell that, while perhaps being infected with a pathogen, cannot or does not supply enough nutrients to the pathogen such that the pathogen can grow and propagate.

A pathogen may be a gram negative bacteria such as: Agrobacterium tumefaciens, Agrobacterium vitis, Burkholderia solanacearum, Burkholderia caryophylli, Erwinia amylovora, Erwinia carotovora, Pseudomonas savastanoi, Pseudomonas syringae, Xanthomonas axonopodis, Xanthomonas campestris, Xantomonas hortorumpelargonium, Xanthomonas oryzae, and Xanthomonas transluceus.

A pathogen may be a gram positive bacteria, such as: Clavibacter michiganensis, Rhodococcus fascians, and Streptomyces scabies.

A pathogen may be a phytopathogenic mould such as: Aspiognomonia veneta, Cryphonectria parasitica, Diaporthe perniciosa, Leucostoma cincta, Cochliobolus sativus, Cochliobolus victoriae, Didymella aplanata, Leptosphaeria maculans, Mycosphaerella arachidicola, Mycosphaerella graminicola, Mycosphaerella musicola Phaesphaeria nodorum, Pyrenophora chaetomioides, Pyrenophora gramine, Pyrenophora teres, Venturia inequalis, Blumeria graminis, Leveillula tauric, Podosphaera leucotricha, Sphaerotheca fuliginia, Uncinula necator, Aspergillus flavus, Penicillium expansum, Claviceps purpurea, Builts black sclerots, Cibberella fujicuroi, Cibberella zeae, Nectria galligena, Diplocarpon rosae, Drepanopeziza ribis, Mollisia acuformis, Pezicula malicortis, Pseudopezicola tracheiphila, Pseudopeziza medicaginis, Magnaporthe grisea, Taphrina deformans, Taphrina pruni, Alternaria solani, Septoria apiicola, Alternaria sp., Aspergillus sp., Aspergillus flavus (which produce aflatoxin B1), Botryodiplodia sp., Botrytis sp., Cercospora musaeis, Cladosporium sp., Colletotrichum sp., Diaporthe sp., Diplodia Fusarium sp., Fusarium oxysporum var. cubense, Geotrichum sp., Gibberella fujikuroi, Gloeosporium sp., Leptosphaeria maculans, Monilia sp., Nigrospora sp., Penicillium sp., Phomopsis sp., Phytophthora sp., Piricularia oryzae, Sclerotinia, Sclerotinia sclerotiorum, Trichoderma sp., and Venturia sp.

The present invention also provides for disease protection, prevention or reducing the likelihood of a plant acquiring a disease by altering the accessibility of an amino acid and/or mineral efflux transporter to a pathogen or a disease caused by a pathogen. The present invention also provides for disease protection, prevention or reducing the likelihood of a plant acquiring a disease by increasing the expression or activity of an amino acid and/or mineral influx transporter in response to a pathogen, thereby directing nutrients away from the pathogen and thus depriving the pathogen of essential nutrition. By way of example, the present invention may protect a plant cell or plant against anthracnose, scab, canker, leaf spot, end rot, brown rot, rust, club root, smut, gall, damping off, dollar spot, mildew, e.g. downy mildew, or powdery mildew, blight, e.g. early blight, late blight, fire blight, fairy rings, wilt (e.g. Fusarium wilt), mold (e.g. gray mold), leaf curl, scab (such as potato scab), verticillium wilt, Anthracnose of Trees, Apple Scab, Artillery Fungus, Azalea Gall, Bacterial Spot of Peach, Bacterial Wilt of Cucurbits, Bark Splitting, Bentgrass Deadspot, Black Knot, Blossom End Rot, Botrytis Blight, Botrytis Blight of Peony, Botrytis Blight of Tulip, Brown Patch, Cane Diseases of Brambles, Canker Diseases of Poplar, Cedar Apple Rust, Cenangium Canker, Clubroot of Cabbage, Corn smut, Cytospora Canker of Fruit, Cytospora Canker of Ornamentals, Daylily Rust, Dog Urine Damage, Dogwood Crown Canker, Downy Leafspot of Hickory, Drechslera Leafspot, Dutch Elm Disease, Fairy Ring, Filbert Blight, Forsythia Gall, Garlic Diseases, Gladiolus Scab, Gray Leafspot, Gray Snow Mold, Hawthorn Leaf Blight, Hemlock Twig Rust, Hollyhock Rust, Juniper Tip Blight, Late Blight, Leaf Tatter, Lilac Bacterial Blight, Oak Leaf Blister, Oedema, Orange Berry Rust, Pachysandra Leaf Blight, Peach Leaf Curl, Physiological Leaf Scorch, Slime Molds, Sphaeropsis (Diplodia), Tar Spot, Tree Cankers, Turfgrass Anthracnose, Willow Black Canker, Willow Botryosphaeria, Willow Leaf Rust, Willow Leucostoma Canker, Willow Powdery Mildew, Willow Scab or Winter Injury.

The present invention provides for protection, prevention or reducing the likelihood that a plant or plant cell will acquire an infectious agent by decreasing the sequestration of an amino acid and/or mineral efflux transporter by a pathogen, thereby depriving the pathogen of essential nutrition. The present invention provides for protection, prevention or reducing the likelihood that a plant or plant cell will acquire an infectious agent by increasing the expression or activity of an amino acid and/or mineral influx transporter in response to a pathogen, thereby directing nutrients away from the pathogen and thus depriving the pathogen of essential nutrition. By way of example infectious agents include: Verticillium fungi, Phragmidium spp., Streptomyces scabies, Taphrina deformans, Phytophthora, Botrytis, Fusarium, Erwinia, Alternaria, Plasmopara, Sclerotinia, Rhizoctonia, Pythium, Agrobacterium, Ustilago, Plasmodiophora, Monilinia, Pseudomonas, Colletotrichum, Puccinia or Tilletia.

By way of example, bacterial pathogens may belong to Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, Phytoplasma and Aspergillus. Nematode pathogens may include Root knot (Meloidogyne spp.); Cyst (Heterodera and Globodera spp.); Root lesion (Pratylenchus spp.); Spiral (Helicotylenchus spp.); Burrowing (Radopholus similis); Bulb and stem (Ditylenchus dipsaci); Reniform (Rotylenchulus reniformis); Dagger (Xiphinema spp.); and Bud and leaf (Aphelenchoides spp.). Parasitic plants may include: Striga, Phoradendron, dwarf mistletoe (Ar-ceuthobium spp.) and dodder (Cuscuta spp.). Broomrape (Orobanche spp.). Examples of molds include slime mold on turfgrass such as either the genera Mucilaga or Physarum.

By way of example, the present invention provides for protection from: Stem rust by Puccinia graminis tritici; Leaf rust by Puccinia recondite; Powdery mildew by Erysiphe graminis tritici; Septoria leaf blotch by Stagonospora nodorum or Septoria nodorum, Stagonospora (Septoria) avenae f. sp. triticea, and Septoria tritici; Spot blotch by Cochliobolus sativus or Helminthosporium sativum; Tan spot by Pyrenophora tritici-repentis; Bacterial blight by Xanthomonas translucens pv. translucens or X. campestris pv. Translucens; Bacterial leaf blight by Pseudomonas syringae pv. Syringae; Heat canker; black point by Cochliobolus sativus or Helminthosporium sativum or related fungi; Ergot by Claviceps purpurea; Glume blotch by Stagonospora nodorum or Septoria nodorum; Loose smut by Ustilago tritici; Scab (head blight) by Fusarium sp. (Gibberella zeae); Stinking smut (bunt) by Tilletia foetida or Tilletia caries; Basal glume rot by Pseudomonas syringae pv. Atrofaciens; Black chaff by Xanthomonas translucens pv. Translucens; Bacterial pink seed by Erwinia rhapontici; Common root rot by Cochliobolus sativus or Helminthosporium sativum; Snow rot and snow mold by Pythium and Fusarium spp.; and Take-all by Gaeumannomyces graminis tritici.

By way of example the crop may be barley. Barley diseases include but are not limited to, Stem rust by Puccinia graminis tritici and Puccinia graminis secalis; Leaf rust by Puccinia hordei; Net blotch by Pyrenophora teres; Powdery mildew by Erysiphe graminis hordei; Scald by Rhynchosporium secalis; Septoria leaf blotch by Stagonospora avenae f. sp. triticea and Septoria passerinii; Spot blotch by Cochliobolus sativus or Helminthosporium sativum; Bacterial blight by Xanthomonas translucens pv. translucens Synonym X. campestris pv. Translucens; Black or semi-loose smut by Ustilago nigra; Covered smut by Ustilago hordei; Black point by Cochliobolus sativus or Helminthosporium sativum or related fungi; Ergot by Claviceps purpurea; Glume blotch by Stagonospora nodorum or Septoria nodorum; Loose smut by Ustilago nuda; Scab (head blight) by Fusarium spp. (Gibberella zeae); Bacterial kernel blights by Pseudomonas syringae pathovars; Black chaff by Xanthomonas translucens pv. Translucens; Common root rot by Cochliobolus sativus or Helminthosporium sativum; and, Take-all by Gaeumannomyces graminis tritici;

By way of example oat diseases include but are not limited to, Stem rust by Puccinia graminis avenae; Crown rust or leaf rust by Puccinia coronate; Bacterial stripe blight by Pseudomonas striafaciens; Black loose smut by Ustilago avenae; Covered smut by Ustilago kolleri; Scab (head blight) by Fusarium spp. (Gibberella zeae); and, Blast by Physiologic disorder;

By way of example, rye diseases include but are not limited to, Stem rust by Puccinia graminis secalis; Leaf rust or brown rust by Puccinia recondita secalis; Tan spot by Pyrenophora tritici-repentis; Ergot by Claviceps purpurea; Scab (head blight) by Fursarium spp. (Gibberella zeae); and, Common root rot and other fungi by Helminthosporium sativum and other fungi.

By way of example, corn disease include but are not limited to, Crazy top by Sclerophthora macrospora; Eyespot by Kabatiella zeae; Northern leaf blight by Helminthosporium turcicum; Rust by Puccinia sorghi; Holcus spot by Pseudomonas syringae; Common Smut by Ustilago maydis; Ear rot by Fusarium moniliforme or Fusarium graminearum; Gibberella stalk rot by Gibberella zeae; Diplodia stalk and ear rot by Diplodia maydis; and, Head smut by Sphacelotheca reiliana.

By way example, diseases to beans include but are not limited to, Rust by Uromyces appendiculatus var. appendiculatus; White mold (sclerotinia rot) by Sclerotinia sclerotiorum; Alternaria blight by Alternaria sp.; Common blight by Xanthomonas campestris pv. Phaseoli; Halo blight by Pseudomonas syringae pv. Phaseolicola; Brown spot by Pseudomonas syringae pv. Syringae; Common blight by Xanthomonas campestris pv. Phaseoli; Halo blight by Pseudomonas syringae pv. Phaseolicola; Brown spot by Pseudomonas syringae pv. Syringae; and, Root rot by Fusarium spp., Rhizoctonia solani, and other fungi.

By way of example diseases to soybean include, but are not limited to, Sclerotinia stem rot (white mold) by Sclerotinia sclerotiorum; Stem canker by Diaporthe phaseolorum var. caulivora; Pod and stem blight by Diaporthe phaseolorum var. sojae; Brown stem rot by Phialophora gregata or Cephalosporium gregatum; Brown spot by Septoria glycines; Downy mildew by Peronospora manshurica; Bacterial blight by Pseudomonas syringae pv. Glycinea; Iron chlorosis by Iron deficiency; Pod and stem blight by Diaporthe phaseolorum var. sojae; Purple stain by Cercospora kikuchii; Fusarium root rot by Fusarium spp.; Phytophthora root rot by Phytophthora sojae; Pythium root rot by Pythium spp.; Rhizoctonia root rot by Rhizoctonia solani; and, Soybean cyst nematode by Heterodera glycines.

By way of example canola (rapeseed) and mustard diseases include but are not limited to, Sclerotinia Stem Rot by Sclerotinia sclerotiorum; Alternaria black spot by Alternaria brassicae and A. raphania White rust by Albugo candida; Blackleg by Leptosphaeria maculans; Downy mildew by Peronospora parasitica; and, Aster yellows by Aster yellows mycoplasm.

By way of example sunflower diseases include but are not limited to, Downy mildew by Plasmopara halstedii; Rust by Puccinia helianthi; Sclerotinia stalk and head rot (white mold) by Sclerotinia sclerotiorum; Verticillium wilt by Verticillium dahlia; Phoma black stem by phoma macdonaldii; Phomopsis stem canker by phomopsis or diaporthe) helianthi; Alternaria leaf and stem spot by Alternaria zinniae and Alternaria helianthi; Septoria leaf spot by Septoria helianthi; Apical chlorosis by Pseudomonas tagetis; Rhizopus head rot by Rhizopus spp.; and, Botrytis head rot by Botrytis cinerea.

By way of example potato diseases include but are not limited to, Soft rot by Erwinia carotovora; RING ROT by Clavibacter sepedonicum; Fusarium dry rot by Fusarium sambucinum or F. sulphureum; Silver scurf by Helminthosporium solani; Blackleg by Erwinia carotovora; Scurf & black canker by Rhizoctonia solani; Early blight by Alternaria solani; Late blight by Phytophthora infestans; Verticillium wilt by Verticillium albo-atrum and V. dahlia; and, Purple top by Aster yellows mycoplasma.

By way of example sugarbeet diseases include, but are not limited to, Bacterial leafspot by Pseudomonas syringae; Cercospora leafspot by Cercospora beticola; sugarbeet powdery mildew by Erysiphe betae; Rhizoctonia root and crown rot by Rhizoctonia solani; and Aphanomyces root rot by Aphonomyces cochlioides.

The present invention also provides methods to prevent accumulation of toxic compounds in a plant cell or plant by controlling pathogen infection. For example inhibiting a pathogen from inducing a host plant to provide a nutrient, such as an amino acid, to the pathogen will prevent accumulation of toxins in crops. By way of further example, Aflatoxin is a term generally used to refer to a group of extremely toxic chemicals produced by two molds, Aspergillus flavus and A. parasiticus. The toxins can be produced when these molds, or fungi, attack and grow on certain plants and plant products.

By way of example, and not as limitation, the pathogen may cause a bacterial disease, which include but are not limited to Bacterial leaf blight (Pseudomonas syringae including subsp. syringae); bacterial mosaic (Clavibacter michiganensis including subsp. tessellarius); Bacterial sheath rot (Pseudomonas fuscovaginae); Basal glume rot (Pseudomonas syringae pv. atrofaciens); Black chaff or bacterial streak (Xanthomonas campestris pv. translucens); Pink seed (Erwinia rhapontici); Spike blight or gummosis (Rathayibacter tritici or Clavibacter tritici, Clavibacter iranicus). The bacterial disease may include Bacterial blight (Pseudomonas amygdali pv. glycinea); Bacterial pustules (Xanthomonas axonopodis pv. glycines or Xanthomonas campestris pv. glycines); Bacterial tan spot (Curtobacterium flaccumfaciens pv. flaccumfaciens or Corynebacterium flaccumfaciens pv. flaccumfaciens); Bacterial wilt (Curtobacterium flaccumfaciens pv. flaccumfaciens); Ralstonia solanacearum or Pseudomonas solanacearum); or Wildfire (Pseudomonas syringae pv. tabaci).

The bacterial diseases include but are not limited to Gumming disease (Xanthomonas campestris pv. vasculorum); Leaf scald (Xanthomonas albilineans); Mottled stripe (Herbaspirillum rubrisubalbicans); Ratoon stunting disease (Leifsonia xyli subsp. xyli); and Red stripe (top rot) (Acidovorax avenae). By further way of example, bacterial pathogens include but are not limited to Bacterial wilt or brown rot (Ralstonia solanacearum or Pseudomonas solanacearum); Blackleg and bacterial soft rot (Pectobacterium carotovorum subsp. Atrosepticum or Erwinia carotovora subsp. Atroseptica or Pectobacterium carotovorum subsp. Carotovorum or E. carotovora subsp. Carotovora or Pectobacterium chrysanthemi or E. chrysanthemi or Dickeya solani); Pink eye (Pseudomonas fluorescens); Ring rot (Clavibacter michiganensis subsp. Sepedonicus or Corynebacterium sepedonicum); Common scab (Streptomyces scabiei or S. scabies or Streptomyces acidiscabies or Streptomyces turgidiscabies); Zebra chip or Psyllid yellows (Candidatus Liberibacter solanacearum); Bacterial streak or black chaff (Xanthomonas campestris pv. Translucens); Halo blight (Pseudomonas coronafaciens pv. Coronafaciens); Bacterial blight (halo blight) (Pseudomonas coronafaciens pv. Coronafaciens); Bacterial stripe blight (Pseudomonas coronafaciens pv. Striafaciens); Black chaff and bacterial streak (stripe) (Xanthomonas campestris pv. Translucens); Bacterial blight (Xanthomonas campestris pv. malvacearum); Crown gall (Agrobacterium tumefaciens); and Lint degradation (Erwinia herbicola or Pantoea agglomerans).

By way of example, and not as limitation, the pathogen may cause a fungal disease, which include but are not limited to Alternaria leaf blight (Alternaria triticina); Anthracnose (Colletotrichum graminicola or Glomerella graminicola [teleomorph]); Ascochyta leaf spot (Ascochyta tritici); Aureobasidium decay (Microdochium bolleyi or Aureobasidium bolleyi); Black head molds or sooty molds (Alternaria spp., Cladosporium spp., Epicoccum spp., Sporobolomyces spp. and Stemphylium spp.); Black point or kernel smudge; Cephalosporium stripe (Hymenula cerealis or Cephalosporium gramineum); Common bunt or stinking smut (Tilletia tritici or Tilletia caries or Tilletia laevis or Tilletia foetida); Common root rot (Cochliobolus sativus [teleomorph], Bipolaris sorokiniana [anamorph], or Helminthosporium sativum); Cottony snow mold (Coprinus psychromorbidus); Crown rot or foot rot, seedling blight, dryland root rot (Fusarium spp., Fusarium pseudograminearum, Gibberella zeae, Fusarium graminearum Group II [anamorph], Gibberella avenacea, Fusarium avenaceum [anamorph], or Fusarium culmorum); Dilophospora leaf spot or twist (Dilophospora alopecuri); Downy mildew or crazy top (Sclerophthora macrospora); Dwarf bunt (Tilletia controversa); Ergot (Claviceps purpurea or Sphacelia segetum [anamorph]); Eyespot or foot rot or strawbreaker (Tapesia yallundae, Ramulispora herpotrichoides [anamorph], or Pseudocercosporella herpotrichoides (W-pathotype), Tapesia acuformis; Ramulispora acuformis [anamorph], or Pseudocercosporella herpotrichoides including var. acuformis R-pathoytpe); False eyespot (Gibellina cerealis); Flag smut (Urocystis agropyri); Foot rot or dryland foot rot (Fusarium spp.); Halo spot (Pseudoseptoria donacis or Selenophoma donacis); Karnal bunt or partial bunt (Tilletia indica or Neovossia indica); Leaf rust or brown rust (Puccinia triticina, Puccinia recondita f.sp. tritici, Puccinia tritici-duri); Leptosphaeria leaf spot (Phaeosphaeria herpotrichoides or Leptosphaeria herpotrichoides or Stagonospora sp. [anamorph]); Loose smut (Ustilago tritici or Ustilago segetum var. tritici, Ustilago segetum var. nuda, Ustilago segetum var. avenae); Microscopica leaf spot (Phaeosphaeria microscopica or Leptosphaeria microscopica); Phoma spot (Phoma spp., Phoma glomerata, Phoma sorghina or Phoma insidiosa); Pink snow mold or Fusarium patch (Microdochium nivale or Fusarium nivale or Monographella nivalis [teleomorph]); Platyspora leaf spot (Clathrospora pentamera or Platyspora pentamera); Powdery mildew (Erysiphe graminis f.sp. tritici, Blumeria graminis, Erysiphe graminis, or Oidium monilioides [anamorph]); Pythium root rot (Pythium aphanidermatum, Pythium arrhenomanes, Pythium graminicola, Pythium myriotylum or Pythium volutum); Rhizoctonia root rot (Rhizoctonia solani); Thanatephorus cucumeris [teleomorph]); Ring spot or Wirrega blotch (Pyrenophora seminiperda, Drechslera campanulata or Drechslera wirreganensis); Scab or head blight (Fusarium spp., Gibberella zeae, Fusarium graminearum Group II [anamorph]; Gibberella avenacea, Fusarium avenaceum [anamorph], Fusarium culmorum, Microdochium nivale, Fusarium nivale, or Monographella nivalis [teleomorph]); Sclerotinia snow mold or snow scald (Myriosclerotinia borealis or Sclerotinia borealis); Sclerotium wilt or Southern blight (Sclerotium rolfsii or Athelia rolfsii [teleomorph]); Septoria blotch (Septoria tritici or Mycosphaerella graminicola [teleomorph]); Sharp eyespot (Rhizoctonia cerealis or Ceratobasidium cereale [teleomorph]); Snow rot (Pythium spp., Pythium aristosporum, Pythium iwayamae or Pythium okanoganense); Southern blight or Sclerotium base rot (Sclerotium rolfsii or Athelia rolfsii [teleomorph]); Speckled snow mold or gray snow mold or Typhula blight (Typhula idahoensis, Typhula incarnata, Typhula ishikariensis or Typhula ishikariensis var. canadensis); Spot blotch (Cochliobolus sativus [teleomorph], Bipolaris sorokiniana [anamorph] or Helminthosporium sativum); Stagonospora blotch (Phaeosphaeria avenaria f.sp. triticae, Stagonospora avenae f.sp. triticae [anamorph], Septoria avenae f.sp. triticea, Phaeosphaeria nodorum, Stagonospora nodorum [anamorph] or Septoria nodorum); Stem rust or black rust (Puccinia graminis, or Puccinia graminis f.sp. tritici (Ug99)); Storage molds (Aspergillus spp. or Penicillium spp.); Stripe rust or yellow rust (Puccinia striiformis or Uredo glumarum [anamorph]); Take-all (Gaeumannomyces graminis var. tritici, Gaeumannomyces graminis var. avenae); Tan spot or yellow leaf spot, red smudge (Pyrenophora tritici-repentis or Drechslera tritici-repentis [anamorph]); Tar spot (Phyllachora graminis or Linochora graminis [anamorph]); or Wheat Blast (Magnaporthe grisea); Zoosporic root rot (Lagena radicicola, Ligniera pilorum, Olpidium brassicae, Rhizophydium graminis). The fungal disease may also include Alternaria leaf spot (Alternaria spp.); Anthracnose (Colletotrichum truncatum, Colletotrichum dematium f. truncatum, Glomerella glycines or Colletotrichum destructivum [anamorph]); Black leaf blight (Arkoola nigra); Black root rot (Thielaviopsis basicola or Chalara elegans [synanamorph]); Brown (Septoria glycines or Mycosphaerella usoenskajae [teleomorph]); Brown stem rot (Phialophora gregata or Cephalosporium gregatum); Charcoal rot (Macrophomina phaseolina); Choanephora leaf blight (Choanephora infundibuliferam or Choanephora trispora); Damping-off (Rhizoctonia solani, Thanatephorus cucumeris [teleomorph], Pythium aphanidermatum, Pythium debaryanum, Pythium irregulare, Pythium myriotylum or Pythium ultimum); Downy mildew (Peronospora manshurica); Drechslera blight (Drechslera glycines); Frogeye leaf spot (Cercospora sojina); Fusarium root rot (Fusarium spp.); Leptosphaerulina leaf spot (Leptosphaerulina trifolii); Mycoleptodiscus root rot (Mycoleptodiscus terrestris); Neocosmospora stem rot (Neocosmospora vasinfecta or Acremonium spp. [anamorph]); Phomopsis seed decay (Phomopsis spp.); Phytophthora root and stem rot (Phytophthora sojae); Phyllosticta leaf spot (Phyllosticta sojaecola); Phymatotrichum root rot or cotton root rot (Phymatotrichopsis omnivora or Phymatotrichum omnivorum); Pod and stem blight (Diaporthe phaseolorum or Phomopsis sojae [anamorph]); Powdery mildew (Microsphaera diffusa); Purple seed stain (Cercospora kikuchii); Pyrenochaeta leaf spot (Pyrenochaeta glycines); Pythium rot (Pythium aphanidermatum or Pythium debaryanum or Pythium irregulare or Pythium myriotylum or Pythium ultimum); Red crown rot (Cylindrocladium crotalariae or Calonectria crotalariae [teleomorph]); Red leaf blotch or Dactuliophora leaf spot (Dactuliochaeta glycines, Pyrenochaeta glycines or Dactuliophora glycines [synanamorph]); Rhizoctonia aerial blight (Rhizoctonia solani or Thanatephorus cucumeris [teleomorph]); Rhizoctonia root and stem rot (Rhizoctonia solani); Rust (Phakopsora pachyrhizi); Scab (Spaceloma glycines); Sclerotinia stem rot (Sclerotinia sclerotiorum); Southern blight (damping-off and stem rot) or Sclerotium blight (Sclerotium rolfsii or Athelia rolfsii [teleomorph]); Stem canker (Diaporthe phaseolorum or Diaporthe phaseolorum var. caulivora or Phomopsis phaseoli [anamorph]); Stemphylium leaf blight (Stemphylium botryosum or Pleospora tarda [teleomorph]); Sudden death syndrome (Fusarium solani f.sp. glycines); Target spot (Corynespora cassiicola); or Yeast spot (Nematospora coryli).

By way of example, fungal diseases also include but are not limited to Anthracnose (Colletotrichum graminicola or Glomerella graminicola [teleomorph]); Blast; Downy mildew (Sclerophthora macrospora); Ergot (Claviceps purpurea or Sphacelia segetum [anamorph]); Fusarium foot rot (Fusarium culmorum); Head blight (Bipolaris sorokiniana or Cochliobolus sativus [teleomorph] or Drechslera avenacea or Fusarium graminearum or Gibberella zeae [teleomorph] or Fusarium spp.); Leaf blotch and crown rot (Helminthosporium leaf blotch) (Drechslera avenacea or Helminthosporium avenaceum or Drechslera avenae or Helminthosporium avenae or Pyrenophora avenae [teleomorph]); Powdery mildew (Erysiphe graminis f. sp. avenae or Erysiphe graminis or Oidium monilioides [anamorph]); Rhizoctonia root rot (Rhizoctonia solani or Thanatephorus cucumeris [teleomorph]); Root rot (Bipolaris sorokiniana or Cochliobolus sativus [teleomorph] or Fusarium spp. or Pythium spp. or Pythium debaryanum or Pythium irregular or Pythium ultimum); Rust, crown (Puccinia coronate); Rust, stem (Puccinia graminis); Seedling blight (Bipolaris sorokiniana or Cochliobolus sativus [teleomorph] or Drechslera avenae or Fusarium culmorum or Pythium spp. or Rhizoctonia solani); Sharp eyespot (Rhizoctonia cerealis or Ceratobasidium cereale [teleomorph]); Smut, covered (Ustilago segetum or Ustilago kolleri); Smut, loose (Ustilago avenae); Snow mold, pink (Fusarium patch) (Microdochium nivale or Fusarium nivale or Monographella nivalis [teleomorph]); Snow mold, speckled or gray (Typhula blight) (Typhula idahoensis or Typhula incarnate or Typhula ishikariensis); Speckled blotch (Septoria blight) (Stagonospora avenae or Septoria avenae or Phaeosphaeria avenaria [teleomorph]); Take-all (white head) (Gaeumannomyces graminis var. avenae or Gaeumannomyces graminis); Victoria blight (Bipolaris victoriae or Cochliobolus victoriae [teleomorph]).

By way of further example, fungal diseases include but are not limited to, Black dot (Colletotrichum coccodes or Colletotrichum atramentarium); Brown spot and Black pit (Alternaria alternate or Alternaria tenuis); Cercospora leaf blotch (Mycovellosiella concors or Cercospora concors or Cercospora solani or Cercospora solani-tuberosi); Charcoal rot (Macrophomina phaseolina or Sclerotium bataticola); Choanephora blight (Choanephora cucurbitarum); Common rust (Puccinia pittieriana); Deforming rust (Aecidium cantensis); Early blight (Alternaria solani); Fusarium dry rot (Fusarium spp. or Gibberella pulicaris or Fusarium solani or Fusarium avenaceum or Fusarium oxysporum or Fusarium culmorum or Fusarium acuminatum or Fusarium equiseti or Fusarium crookwellense); Fusarium wilt (Fusarium spp. or Fusarium avenaceum or Fusarium oxysporum or Fusarium solani f.sp. eumartii); Gangrene (Phoma solanicola f. foveata or Phoma foveata or Phoma exigua var. foveata or Phoma exigua f. sp. Foveata or Phoma exigua var. exigua); Gray mold (Botrytis cinerea); Late blight (Phytophthora infestans); Leak (Pythium spp. or Pythium ultimum var. ultimum or Pythium debaryanum or Pythium aphanidermatum or Pythium deliense); Phoma leaf spot (Phoma andigena var. andina); Pink rot (Phytophthora spp. or Phytophthora cryptogea or Phytophthora drechsleri or Phytophthora erythroseptica or Phytophthora megasperma or Phytophthora nicotianae var. parasitica); Powdery mildew (Erysiphe cichoracearum); Powdery scab (Spongospora subterranea f.sp. subterranean); Rhizoctonia canker and black scurf (Rhizoctonia solani or Thanatephorus cucumeris [teleomorph]); Rosellinia black rot (Rosellinia sp. or Dematophora sp. [anamorph]); Septoria leaf spot (Septoria lycopersici var. malagutii); Silver scurf (Helminthosporium solani); Skin spot (Polyscytalum pustulans); Stem rot (southern blight) (Sclerotium rolfsii or Athelia rolfsii [teleomorph]); Thecaphora smut (Angiosorus solani or Thecaphora solani); Ulocladium blight (Ulocladium atrum); Verticillium wilt (Verticillium albo-atrum or Verticillium dahlia); Wart (Synchytrium endobioticum); and, White mold (Sclerotinia sclerotiorum).

Fungal diseases also include but are not limited to, Anthracnose (Colletotrichum graminicola or Glomerella graminicola [teleomorph]); Black head molds (Alternaria spp. or Cladosporium herbarum or Mycosphaerella tassiana [teleomorph] or Epicoccum spp. or Sporobolomyces spp. or Stemphylium spp.); Black point (Bipolaris sorokiniana or Cochliobolus sativus [teleomorph] or Fusarium spp.); Bunt or stinking smut (Tilletia caries or Tilletia tritici or Tilletia laevis or Tilletia foetida); Cephalosporium stripe (Hymenula cerealis or Cephalosporium gramineum); Common root rot and seedling blight (Bipolaris sorokiniana or Helminthosporium sativum or Cochliobolus sativus [teleomorph]); Cottony snow mold or winter crown rot (Coprinus psychromorbidus); Dilophospora leaf spot (twist) (Dilophospora alopecuri); Dwarf bunt (Tilletia controversa); Ergot (Claviceps purpurea or Sphacelia segetum [anamorph]); Fusarium root rot (Fusarium culmorum); Halo spot (Pseudoseptoria donacis or Selenophoma donacis); Karnal bunt (partial bunt) (Neovossia indica or Tilletia indica); Leaf rust (brown rust) (Puccinia recondite or Aecidium clematidis [anamorph]); Leaf streak (Cercosporidium graminis or Scolicotrichum graminis); Leptosphaeria leaf spot (Phaeosphaeria herpotrichoides or Leptosphaeria herpotrichoides); Loose smut (Ustilago tritici); Pink snow mold (Fusarium patch) (Microdochium nivale or Fusarium nivale or Monographella nivalis [teleomorph]); Powdery mildew (Erysiphe graminis or Pythium root rot or Pythium aphanidermatum or Pythium arrhenomanes or Pythium debaryanum or Pythium graminicola or Pythium ultimum); Scab (Gibberella zeae or Fusarium graminearum [anamorph]); Septoria leaf blotch (Septoria secalis); Septoria tritici blotch (speckled leaf blotch) (Septoria tritici or Mycosphaerella graminicola [teleomorph]); Sharp eyespot and Rhizoctonia root rot (Rhizoctonia cerealis or Ceratobasidium cereale [teleomorph]); Snow scald (Sclerotinia snow mold) (Myriosclerotinia borealis or Sclerotinia borealis); Speckled (or gray) snow mold (Typhula blight) (Typhula idahoensis or Typhula incarnate or Typhula ishikariensis or Typhula ishikariensis var. Canadensis); Spot blotch (Bipolaris sorokiniana); Stagonospora blotch (glume blotch) (Stagonospora nodorum or Septoria nodorum or Phaeosphaeria nodorum [teleomorph] or Leptosphaeria nodorum); Stalk smut (stripe smut) (Urocystis occulta); Stem rust (Puccinia graminis); Storage molds (Alternaria spp. or Aspergillus spp. or Epicoccum spp. or Nigrospora spp. or Penicillium spp. or Rhizopus spp.); Strawbreaker (eyespot or foot rot) (Pseudocercosporella herpotrichoides or Tapesia acuformis [teleomorph]); Stripe rust (yellow rust) (Puccinia striiformis or Uredo glumarum [anamorph]); Take-all (Gaeumannomyces graminis); Tan spot (yellow leaf spot) (Pyrenophora tritici-repentis or Drechslera tritici-repentis [anamorph] or Helminthosporium tritici-repentis).

Fungal diseases also include but are not limited to Alternaria leaf blight (Alternaria tenuissima); Alternaria leaf spot (Alternaria arachidis); Alternaria spot and veinal necrosis (Alternaria alternate); Anthracnose (Colletotrichum arachidis or Colletotrichum dematium or Colletotrichum mangenoti); Aspergillus crown rot (Aspergillus niger); Blackhull (Thielaviopsis basicola or Chalara elegans [synanamorph]); Botrytis blight (Botrytis cinerea or Botryotinia fuckeliana [teleomorph]); Charcoal rot and Macrophomina leaf spot (Macrophomina phaseolina or Rhizoctonia bataticola); Choanephora leaf spot (Choanephora spp.); Collar rot (Lasiodiplodia theobromae or Diplodia gossypina); Colletotrichum leaf spot (Colletotrichum gloeosporioides or Glomerella cingulata [teleomorph]); Cylindrocladium black rot (Cylindrocladium crotalariae or Calonectria crotalariae [teleomorph]); Cylindrocladium leaf spot (Cylindrocladium scoparium or Calonectria kyotensis [teleomorph]); Damping-off, Aspergillus (Aspergillus flavus or Aspergillus niger); Damping-off, Fusarium (Fusarium spp.); Damping-off, Pythium (Pythium spp.); Damping-off, Rhizoctonia (Rhizoctonia spp.); Damping-off, Rhizopus (Rhizopus spp.); Drechslera leaf spot (Bipolaris spicifera or Drechslera spicifera or Cochliobolus spicifer [teleomorph]); Fusarium peg and root rot (Fusarium spp.); Fusarium wilt (Fusarium oxysporum); Leaf spot, early (Cercospora arachidicola or Mycosphaerella arachidis [teleomorph]); Leaf spot, late (Phaeoisariopsis personata or Cercosporidium personatum or Mycosphaerella berkeleyi [teleomorph]); Melanosis (Stemphylium botryosum or Pleospora tarda [teleomorph]); Myrothecium leaf blight (Myrothecium roridum); Olpidium root rot (Olpidium brassicae); Pepper spot and scorch (Leptosphaerulina crassiasca); Pestalotiopsis leaf spot (Pestalotiopsis arachidis); Phoma leaf blight (Phoma microspora); Phomopsis foliar blight (Phomopsis phaseoli or Phomopsis sojae or Diaporthe phaseolorum [teleomorph]); Phomopsis leaf spot (Phomopsis spp.); Phyllosticta leaf spot (Phyllosticta arachidis-hypogaeae or Phyllosticta sojaecola or Pleosphaerulina sojicola [teleomorph]); Phymatotrichum root rot (Phymatotrichopsis omnivore or Phymatotrichum omnivorum); Pod rot (pod breakdown) (Fusarium equiseti or Fusarium scirpi or Gibberella intricans [teleomorph] or Fusarium solani or Nectria haematococca [teleomorph] or Pythium myriotylum or Rhizoctonia solani or Thanatephorus cucumeris [teleomorph]); Powdery mildew (Oidium arachidis); Pythium peg and root rot (Pythium myriotylum or Pythium aphanidermatum or Pythium debaryanum or Pythium irregular or Pythium ultimum); Pythium wilt (Pythium myriotylum); Rhizoctonia foliar blight, peg and root rot (Rhizoctonia solani); Rust (Puccinia arachidis); Scab (Sphaceloma arachidis); Sclerotinia blight (Sclerotinia minor or Sclerotinia sclerotiorum); Stem rot (southern blight) (Sclerotium rolfsii or Athelia rolfsii [teleomorph]); Verticillium wilt (Verticillium albo-atrum or Verticillium dahlia); Web blotch (net blotch) (Phoma arachidicola or Ascochyta adzamethica or Didymosphaeria arachidicola or Mycosphaerella arachidicola); Yellow mold (Aspergillus flavus or Aspergillus parasiticus); Zonate leaf spot (Cristulariella moricola or Sclerotium cinnamomi [syanamorph] or Grovesinia pyramidalis [teleomorph]).

Fungal diseases also include but are not limited to Anthracnose (Glomerella gossypii or Colletotrichum gossypii [anamorph]); Areolate mildew (Ramularia gossypii or Cercosporella gossypii or Mycosphaerella areola [teleomorph]); Ascochyta blight (Ascochyta gossypii); Black root rot (Thielaviopsis basicola or Chalara elegans [synanamorph]); Boll rot (Ascochyta gossypii or Colletotrichum gossypii or Glomerella gossypii [teleomorph] or Fusarium spp. or Lasiodiplodia theobromae or Diplodia gossypina or Botryosphaeria rhodina [teleomorph] or Physalospora rhodina or Phytophthora spp. or Rhizoctonia solani); Charcoal rot (Macrophomina phaseolina); Escobilla (Colletotrichum gossypii or Glomerella gossypii [teleomorph]); Fusarium wilt (Fusarium oxysporum f.sp. vasinfectum); Leaf spot (Alternaria macrospora or Alternaria alternata or Cercospora gossypina or Mycosphaerella gossypina [teleomorph] or Cochliobolus spicifer or Bipolaris spicifera [anamorph] or Curvularia spicifera or Cochliobolus spicifer or Myrothecium roridum or Rhizoctonia solani or Stemphylium solani); Lint contamination (Aspergillus flavus or Nematospora spp. or Nigrospora oryzae); Phymatotrichum root rot or cotton root rot (Phymatotrichopsis omnivora or Phymatotrichum omnivorum); Powdery mildew (Leveillula taurica or Oidiopsis sicula [anamorph] or Oidiopsis gossypii or Salmonia malachrae); Stigmatomycosis (Ashbya gossypii or Eremothecium coryli or Nematospora coryli or Aureobasidium pullulans); Cotton rust (Puccinia schedonnardii); Southwestern cotton rust (Puccinia cacabata); Tropical cotton rust (Phakopsora gossypii); Sclerotium stem and root rot or southern blight (Sclerotium rolfsii or Athelia rolfsii [teleomorph]); Seedling disease complex (Colletotrichum gossypii or Fusarium spp. or Pythium spp. or Rhizoctonia solani or Thanatephorus cucumeris [teleomorph] or Thielaviopsis basicola or Chalara elegans [synanamorph]); Stem canker (Phoma exigua); and Verticillium wilt (Verticillium dahliae).

The fungal disease may also include but are not limited to Banded sclerotial (leaf) disease (Thanatephorus cucumeris or Pellicularia sasakii or Rhizoctonia solani [anamorph]); Black rot (Ceratocystis adiposa or Chalara sp. [anamorph]); Black stripe (Cercospora atrofiliformis); Brown spot (Cercospora longipes); Brown stripe (Cochliobolus stenospilus or Bipolaris stenospila [anamorph]); Downy mildew (Peronosclerospora sacchari or Sclerospora sacchari); Downy mildew, leaf splitting form (Peronosclerospora miscanthi or Sclerospora mischanthi or Mycosphaerella striatiformans); Eye spot (Bipolaris sacchari or Helminthosporium sacchari); Fusarium sett and stem rot (Gibberella fujikuroi or Fusarium moniliforme [anamorph] or Gibberella subglutinans); iliau (Clypeoporthe iliau or Gnomonia iliau or Phaeocytostroma iliau [anamorph]); Leaf blast (Didymosphaeria taiwanensis); Leaf blight (Leptosphaeria taiwanensis or Stagonospora tainanensis [anamorph]); Leaf scorch (Stagonospora sacchari); Marasmius sheath and shoot blight (Marasmiellus stenophyllus or Marasmius stenophyllus); Myriogenospora leaf binding (tangle top) (Myriogenospora aciculispora); Phyllosticta leaf spot (Phyllosticta hawaiiensis); Phytophthora rot of cuttings (Phytophthora spp. or Phytophthora megasperma); Pineapple disease (Ceratocystis paradoxa or Chalara paradoxa or Thielaviopsis paradoxa [anamorph]); Pokkah boeng (Gibberella fujikuroi or Fusarium moniliforme [anamorph] or Gibberella subglutinans); Red leaf spot (purple spot) (Dimeriella sacchari); Red rot (Glomerella tucumanensis or Physalospora tucumanensis or Colletotrichum falcatum [anamorph]); Red rot of leaf sheath and sprout rot (Athelia rolfsii or Pellicularia rolfsii or Sclerotium rolfsii [anamorph]); Red spot of leaf sheath (Mycovellosiella vaginae or Cercospora vaginae); Rhizoctonia sheath and shoot rot (Rhizoctonia solani); Rind disease (sour rot) (Phaeocytostroma sacchari or Pleocyta sacchari or Melanconium sacchari); Ring spot (Leptosphaeria sacchari or Phyllosticta sp. [anamorph]); Root rot (Marasmius sacchari or Pythium arrhenomanes or Pythium graminicola or Rhizoctonia sp. or Oomycetes); common Rust (Puccinia melanocephala or Puccinia erianthi); Orange Rust (Puccinia kuehnii); Schizophyllum rot (Schizophyllum commune); Sclerophthora disease (Sclerophthora macrospora); Seedling blight (Alternaria alternata or Bipolaris sacchari or Cochliobolus hawaiiensis or Bipolaris hawaiiensis [anamorph] or Cochliobolus lunatus or Curvularia lunata [anamorph] or Curvularia senegalensis or Setosphaeria rostrata or Exserohilum rostratum [anamorph] or Drechslera halodes); Sheath rot (Cytospora sacchari); Smut, culmicolous (Ustilago scitaminea); Target blotch (Helminthosporium sp.); Veneer blotch (Deightoniella papuana); White rash (Elsinoe sacchari or Sphaceloma sacchari [anamorph]); Wilt (Fusarium sacchari or Cephalosporium sacchari); Yellow spot (Mycovellosiella koepkei or Cercospora koepkei); Zonate leaf spot (Gloeocercospora sorghi); Lesion (Pratylenchus spp.); Root-knot (Meloidogyne spp.); Spiral (Helicotylenchus spp. or Rotylenchus spp. or Scutellonema spp.).

The pathogen may be a phytoplasma such as aster yellows phytoplasma, Cowpea mild mottle, Groundnut crinkle, Groundnut eyespot, Groundnut rosette, Groundnut chlorotic rosette, Groundnut green rosette, Groundnut streak, Marginal chlorosis, Peanut clump, Peanut green mosaic, Peanut mottle, Peanut ringspot or bud necrosis, Tomato spotted wilt, Peanut stripe, Peanut stunt, Peanut yellow mottle, Tomato spotted wilt, or Witches' broom.

By way of example nematode pathogens include but are not limited to, Potato cyst nematode, Globodera rostochiensis, Globodera pallid, Lesion nematode, Pratylenchus spp., Pratylenchus brachyurus, Pratylenchus penetrans, Pratylenchus scribneri, Pratylenchus neglectus, Pratylenchus thornei, Pratylenchus crenatus, Pratylenchus andinus, Pratylenchus vulnus, Pratylenchus coffeae, Potato rot nematode, Ditylenchus destructor, Root knot nematode, Meloidogyne spp., Meloidogyne hapla, Meloidogyne incognita, Meloidogyne javanica, Meloidogyne chitwoodi, Sting nematode, Belonolaimus longicaudatus, Stubby-root nematode, Paratrichodorus spp., Trichodorus spp; Heterodera avenae, Ditylenchus dipsaci, Subanguina radicicola, Meloidogyne spp., Anguina tritici, Xiphinema spp., Tylenchorhynchus brevilineatus, Tylenchorhynchus brevicadatus, Criconemella ornate, Macroposthonia ornate, Meloidogyne javanica, Meloidogyne hapla, Meloidogyne arenaria, Pratylenchus brachyurus, Pratylenchus coffeae, Ditylenchus destructor, Scutellonema cavenessi, Belonolaimus glacilis, Belonolaimus longicaudatus, Ditylenchus dipsaci, Heterodera avenae, Heterodera hordecalis, Heterodera latipons, Punctodera chalcoensis, Xiphinema americanum, Pratylenchus spp., Pratylenchus thornei, Pratylenchus spp., Criconemella spp., Nothocriconemella mutabilis, Meloidogyne spp., Meloidogyne chitwoodi, Meloidogyne naasi, Hemicycliophora spp., Helicotylenchus spp., Belonolaimus longicaudatus, Paratrichodorus minor, Quinisulcius capitatus, Tylenchorhynchus spp., and Merlinius spp., Hoplolaimus columbus, Rotylenchulus reniformis, Meloidogyne incognita, Belonolaimus longicaudatus, and Aphelenchoides arachidis.

The present invention provides for plant cells that are resistant to pathogens. In one embodiment, the plant cells comprise at least one copy of a gene encoding an amino acid and/or mineral efflux transporter that is modified or mutated such that the overall activity of expression of the amino acid and/or mineral efflux transporter is decreased as compared to unmodified plants. In another embodiment, the plant cells comprise a genetic such that the overall activity of expression of the amino acid and/or mineral efflux transporter is increased as compared to unmodified plants. In certain specific embodiments, the genetic mutation to increase the overall activity of expression of the amino acid and/or mineral efflux transporter comprises one or more additional copies of the efflux transporter gene inserted into the plant cells. In another embodiment, the plant cells comprise at least one copy of a gene encoding an amino acid and/or mineral influx transporter such that the overall activity of expression of the amino acid and/or mineral influx transporter is increased as compared to unmodified plants. In certain specific embodiments, the genetic mutation to increase the overall activity of expression of the amino acid and/or mineral influx transporter comprises one or more additional copies of the influx transporter gene inserted into the plant cells. In another embodiment, the plant cells comprise at least one copy of a gene encoding an amino acid and/or mineral influx transporter that is modified or mutated such that the overall activity of expression of the amino acid and/or mineral influx transporter is decreased as compared to unmodified plants.

As used herein, the term “gene” means a stretch of nucleotides that encode a polypeptide. The “gene,” for the purposes of the present invention, need not have introns and regulatory regions associated with the coating region. Accordingly, a cDNA that encodes a polypeptide is considered a “gene” for the purposes of the present invention. Of course, the term “gene” also includes the full length polynucleotide, or any portion thereof, that encodes a polypeptide and may or may not include introns, promoters, enhancers, UTRs, etc.

A genetic modification may be to the coding region for expressing the efflux transporter protein or to the influx transporter protein or to a region involved in affecting expression and/or the functional activity of the efflux transporter protein or influx transporter protein. The modification may be an insertion, a deletion, or a replacement of nucleic acids into the gene that normally encodes the amino acid and/or mineral efflux transporter or into the gene that normally encodes the amino acid and/or mineral influx transporter. The modification may also include replacement of the gene that normally encodes the amino acid and/or mineral efflux transporter with a mutant gene that encodes a modified amino acid and/or mineral efflux transporter via homologous recombination. The modification may also include introducing additional copies of the gene that normally encodes the amino acid and/or mineral influx transporter. The modification may include the introduction of an additional peptide, such as a targeting peptide or a functional domain from another protein, which decreases the activity of the amino acid and/or mineral efflux transporter protein in a particular cell type. The modification may include the introduction of an additional peptide, such as a targeting peptide or a functional domain from another protein, which increases the activity or expression of the amino acid and/or mineral influx transporter protein in particular cell type.

The modification may be a mutation to a regulatory domain such as a promoter or other 5′ or 3′ untranslated domain. The modification may be to a promoter, a coding region, an intron of the gene, a splice site of the gene or an exon of the gene. The modification may be a point mutation, a silent mutation, an insertion or a deletion. An insertion or a deletion may be any number of nucleic acids, and the invention is not limited by the number of additions or deletions that effectuate the genetic modification. In one embodiment, the modification to the efflux transporter should decrease or reduce the ability of the efflux transporter to transport or sense a nutrient. In another embodiment, the modification to the influx transporter should increase or enhance the ability of the influx transporter to transport or sense a nutrient. Accordingly, the modification may occur at the biogenesis of the efflux transporter or influx transporter at the genetic level from promoter to posttranslational modification, as well as at the level of affecting turnover and inactivation, e.g., by phosphorylation or ubiquitination (see, e.g., Niittylae et al. Mol Cell Proteomics, 6(10):1711-26 (2007)). For example, disruption of a site for post-translational modification, such as a site for phosphorylation or ubiquitination, may provide a suitable modification to disrupt the functioning of the transporter.

In one embodiment, the present invention provides methods of regulating an amino acid and/or mineral efflux transporter expression by modifying an amino acid and/or mineral efflux transporter gene. In one embodiment, inserting or introducing one ineffective (or less effective) copy of an efflux transporter may be sufficient to inhibit or reduce the function of an efflux transporter, if the efflux transporter normally exists as a multimer. In another embodiment, inserting one additional copy of an efflux transporter may be sufficient to increase the expression or function of an efflux transporter, if the efflux transporter normally exists as a multimer. The gene encoding the amino acid and/or mineral efflux transporter may be modified upstream of the coding region, such as in a transcription factor binding site, such as a TAL effector. The binding site may be modified by mutating a repeat sequence upstream of the coding region. As discussed herein, mutations may include insertion or deletion of one or several nucleic acids. Mutations may also include the replacement of a region with that of another region, such as a promoter for a tissue specific promoter or a transcription binding factor domain with that of a second transcription factor binding domain.

In another embodiment, the present invention provides methods of regulating an amino acid and/or mineral influx transporter expression by modifying the expression levels or activity an amino acid and/or mineral influx transporter gene. In one mebodiment, inserting or introducing one ineffective (or less effective) copy of an influx transporter may be sufficient to inhibit or reduce the function of an influx transporter, if the influx transporter normally exists as a multimer. In one embodiment, inserting one additional copy of an influx transporter may be sufficient to increase the expression or function of an influx transporter, if the influx transporter normally exists as a multimer. The gene encoding the amino acid and/or mineral influx transporter may be modified upstream of the coding region, such as in a transcription factor binding site, such as a TAL effector, such that transcription of the influx transporter in increased. As discussed herein, mutations may include insertion or deletion of one or several nucleic acids. Mutations may also include the replacement of a region with that of another region, such as a promoter for a tissue specific promoter or a transcription binding factor domain with that of a second transcription factor binding domain.

The present invention provides for affecting the transport of nutrients that interact with an amino acid and/or mineral efflux transporters. The present invention also provides for affecting the transport of nutrients that interact with an amino acid and/or mineral influx transporters. The interacting nutrient may be a ligand, which may refer to a molecule or a substance that can bind to a protein such as a periplasmic binding protein to form a complex with that protein. The binding of the ligand to the protein may distort or change the shape of the protein, particularly the tertiary and quaternary structures.

In one embodiment, the present invention provides for introducing exogenous nucleic acids encoding an amino acid and/or mineral efflux transporter protein into a plant cell. In another embodiment, the present invention provides for introducing exogenous nucleic acids encoding an amino acid and/or mineral influx transporter protein into a plant cell. The introduced exogenous nucleic acids may be intended to be expressed as a mutant protein or wild-type protein. As used herein, an exogenous nucleic acid is a polynucleotide that normally does not exist or occur in the genome of the plant cell. For example, an extra copy of polynucleotide encoding a wild-type influx or efflux transporter would be an exogenous nucleic acid. Of course copies of polynucleotides encoding mutant efflux or influx transporters would also be considered an exogenous nucleic acid. As used herein with respect to proteins and polypeptides, the term “recombinant” may include proteins and/or polypeptides and/or peptides that are produced or derived by genetic engineering, for example by translation in a cell of non-native nucleic acid or that are assembled by artificial means or mechanisms.

The present invention provides for nutrient efflux or influx transporters operably linked with other nucleic acids encoding peptides intended to alter the expression, activity or location of the efflux or influx transporter, such as targeting sequences. As used herein, fusion may refer to nucleic acids and polypeptides that comprise sequences that are not found naturally associated with each other in the order or context in which they are placed according to the present invention. A fusion nucleic acid or polypeptide does not necessarily comprise the natural sequence of the nucleic acid or polypeptide in its entirety. In general, fusion proteins have the two or more segments joined together through normal peptide bonds. Fusion nucleic acids have the two or more segments joined together through normal phosphodiester bonds.

In one embodiment, the present invention provides for decreasing expression of an amino acid and/or mineral nutrient efflux transporter post-transcriptionally. In another embodiment, the present invention provides for decreasing expression of an amino acid and/or mineral nutrient influx transporter post-transcriptionally. In certain embodiments embodiment, antisense technology or RNAi technology can be used to reduce expression of an efflux or influx transporter protein. These techniques are well known. For example, a single-stranded RNA that can hybridize to an mRNA transcript transcribed from an endogenous efflux or influx transporter gene can be introduced into the cell to interfere with translation. Alternatively, dsRNA containing a region of perfect or significant nucleotide sequence identity with an mRNA transcript transcribed from an endogenous efflux or influx transporter gene, and containing the complement thereto, can be introduced into the cell to interfere with translation by inducing RNAi through well-known principles. Alternatively, the plant cell may be contacted with an antibody or fragment directed against the efflux or influx transporter. As used herein, the term dsRNA refers to double-stranded RNA, wherein the dsRNA may be two separate strands or may be a single strand that folds back on itself in a self-complementary fashion to form a hairpin loop. The dsRNA used in the methods and plant cells of the present invention may comprise a nucleotide sequence identical or nearly identical to the nucleotide of a target gene such that expression of the target gene is specifically downregulated. dsRNA may be produced by expression vectors (also referred to as RNAi expression vectors) capable of giving rise to transcripts which form self-complementary dsRNAs, such as hairpin RNAs, or dsRNA formed by separate complementary RNA strands in cells, and/or transcripts which can produce siRNAs in vivo. Vectors may include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops, which in their vector form are not bound to the chromosome.

The genetic modifications used in the methods of the present invention or present in the plant cells of the present invention may comprise more than one modification. For example, the expression or activity of more than one efflux or influx transporter may be modified according to the methods of the present invention. Alternatively, more than one modification may be performed on a single influx or efflux transporter. For example, a genetic construct encoding a hairpin dsRNA may be inserted into a plant cell. The hairpin dsRNA might be designed to reduce expression of an endogenous efflux or influx transporter by designing the nucleotide sequence of the dsRNA to correspond to the 3′ UTR of the endogenous efflux or influx transporter mRNA. Additionally, another genetic construct might be inserted into the same plant cell containing the dsRNA construct, and this additional construct might code for a mutant version of the same efflux or influx transporter, respectively, where the mutant version of the efflux or influx transporter is designed not to include a 3′ UTR, e.g., a cDNA, such that the dsRNA would not be able to interfere with the expression of the mutant efflux or influx transporter gene. In this manner, the expression of activity of the endogenous (or normal) amino acid and/or mineral efflux or influx transporter would be reduced in the genetically modified plant cell compared to an unmodified plant cell.

Similarly, in one embodiment of the present invention, a genetic construct encoding a hairpin dsRNA may be inserted into a plant cell. The hairpin dsRNA might be designed to reduce expression of an endogenous efflux or influx transporter by designing the nucleotide sequence of the dsRNA to correspond to the 3′ UTR of the endogenous efflux or influx transporter mRNA. Additionally, another genetic construct might be inserted into the same plant cell containing the dsRNA construct, and this additional construct might code for a normal version of the same efflux or influx transporter, except that the promoter driving expression of the exogenous copy of the efflux or influx transporter gene would be replaced with a promoter that the pathogen is not be able to manipulate. The exogenous copy of the efflux or influx transporter gene with the “mismatched” promoter may or may not be designed to exclude a 3′ UTR, e.g., a cDNA, such that the dsRNA would not be able to interfere with the expression of the exogenous efflux or influx transporter gene. In this manner, the expression of activity of the endogenous (or normal) amino acid and/or mineral efflux or influx transporter would be reduced in the genetically modified plant cell compared to an unmodified plant cell.

In addition, the invention also provides for more than one modification such that expression or activity of one of more endogenous amino acid and/or mineral efflux transporter is altered (increased or decreased) while expression or activity of one or more endogenous amino acid and/or mineral influx transporter is also altered (increased or decreased). In other embodiments, methods of altering (increasing or decreasing) expression or activity of transporter proteins (influx or efflux) and methods of altering (increasing or decreasing) expression of an endogenous transporter proteins (influx or efflex) can be applied to the same cells or tissues or to different cells or tissues within the same plant.

The present invention provides for methods of altering the expression or functioning of an amino acid and/or mineral efflux or influx transporter, either in the transporter itself or in regulatory element within the gene of the transporter. A transporter may be isolated. As used herein, the term isolated refers to molecules separated from other cell/tissue constituents (e.g. DNA or RNA) that are present in the natural source of the macromolecule. The term isolated may also refer to a nucleic acid or peptide that is substantially free of cellular material, viral material, and culture medium when produced by recombinant DNA techniques, or that is substantially free of chemical precursors or other chemicals when chemically synthesized. Moreover, an isolated nucleic acid may include nucleic acid fragments which are not naturally occurring as fragments and would not be found in the natural state.

An expression vector is one into which a desired nucleic acid sequence may be inserted by restriction and ligation such that it is operably joined or operably linked to regulatory sequences and may be expressed as an RNA transcript. Expression refers to the transcription and/or translation of an endogenous gene, transgene or coding region in a cell.

A coding sequence and regulatory sequences are operably joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

Vectors may further contain one or more promoter sequences. A promoter may include an untranslated nucleic acid sequence usually located upstream of the coding region that contains the site for initiating transcription of the nucleic acid. The promoter region may also include other elements that act as regulators of gene expression. In further embodiments of the invention, the expression vector contains an additional region to aid in selection of cells that have the expression vector incorporated. The promoter sequence is often bounded (inclusively) at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Activation of promoters may be specific to certain cells or tissues, for example by transcription factors only expressed in certain tissues, or the promoter may be ubiquitous and capable of expression in most cells or tissues.

A promoter also optionally includes distal enhancer or repressor elements, which can be located as much as several thousand base pairs from the start site of transcription. A constitutive promoter is a promoter that is active under most environmental and developmental conditions. An inducible promoter is a promoter that is active under environmental or developmental regulation. Any inducible promoter can be used, see, e.g., Ward et al. Plant Mol. Biol. 22:361-366, 1993. Exemplary inducible promoters include, but are not limited to, that from the ACEI system (responsive to copper) (Meft et al. Proc. Natl. Acad. Sci. USA 90:4567-4571, 1993; Int gene from maize (responsive to benzenesulfonamide herbicide safeners) (Hershey et al. Mol. Gen. Genetics 227:229-237, 1991, and Gatz et al. Mol. Gen. Genetics 243:32-38, 1994) or Tet repressor from Tn10 (Gatz et al. Mol. Gen. Genetics 227:229-237, 1991). The inducible promoter may respond to an agent foreign to the host cell, see, e.g., Schena et al. PNAS 88: 1042140425, 1991.

In one embodiment, the modified amino acid and/or mineral efflux transporters of the present invention may function properly in at least one tissue and may function improperly in at least one tissue. For example, introducing a modified efflux transporter with a tissue specific promoter may provide for modified efflux transporter expression in particular tissues (e.g. leaf), leaving a functioning endogenous copy of an efflux transporter in other tissues (e.g. root). In another embodiment, additional copies of at least one amino acid and/or mineral influx transporter of the present invention may function properly in at least one tissue and may not function in at least one other tissue. For example, introducing additional copies of an influx transporter gene with a tissue specific promoter may provide for increased influx transporter expression in particular tissues (e.g. leaf), while not altering the expression levels of the same influx transporter in other tissues (e.g. root). The present invention thus provides for directed expression of nucleic acids encoding efflux transporters, influx transporters, modified efflux transporters and/or modified influx transporters. It is known in the art that expression of a gene can be regulated through the presence of a particular promoter upstream (5′) of the coding nucleotide sequence. Tissue specific promoters for directing expression in plants are known in the art. For example, promoters that direct expression in the roots, seeds, or fruits are known. The promoter may be tissue-specific or tissue-preferred promoters. A tissue specific promoter assists to produce the modified efflux transporter or additional influx transporter exclusively, or preferentially, in a specific tissue. Any tissue-specific or tissue-preferred promoter can be utilized. In plant cells, for example but not by way of limitation, tissue-specific or tissue-preferred promoters include, a root-preferred promoter such as that from the phaseolin gene (Murai et al. Science 23: 476-482, 1983, and Sengupta-Gopalan et al. PNAS 82: 3320-3324, 1985); a leaf-specific and light-induced promoter such as that from cab or rubisco (Simpson et al. EMBO J. 4(11): 2723-2729, 1985, and Timko et al. Nature 318: 579-582, 1985); an anther-specific promoter such as that from LAT52 (Twell et al. Mol. Gen. Genetics 217: 240-245, 1989); a pollen-specific promoter such as that from Zm13 (Guerrero et al. Mol. Gen. Genetics 244: 161-168, 1993) or a microspore-preferred promoter such as that from apg (Twell et al. Sex. Plant Reprod. 6: 217-224, 1993).

In the alternative, the promoter may or may not be a constitutive promoter. Contitutive promoters include, but are not limited to, promoters from plant viruses such as the 35S promoter from CaMV (Odell et al. Nature 313: 810-812, 1985) and the promoters from such genes as rice actin (McElroy et al. Plant Cell 2: 163-171, 1990); ubiquitin (Christensen et al. Plant Mol. Biol. 12:619-632, 1989, and Christensen et al. Plant Mol. Biol. 18: 675-689, 1992); pEMU (Last et al. Theor. Appl. Genet. 81:581-588, 1991); MAS (Velten et al. EMBO J. 3:2723-2730, 1984) and maize H3 histone (Lepetit et al. Mol. Gen. Genetics 231: 276-285, 1992 and Atanassova et al. Plant Journal 2(3): 291-300, 1992).

Vectors may further contain one or more marker sequences suitable for use in the identification and selection of cells which have been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Vectors may be those capable of autonomous replication and expression of the structural gene products present in the DNA segments to which they are operably joined.

The present invention provides for assembling an amino acid and/or mineral efflux transporter or an amino acid and/or mineral influx transporter with another peptide, typically by fusing different nucleic acids together so that they are operably linked and express a fusion protein or a chimeric protein. As used herein, the term fusion protein or chimeric protein may refer to a polypeptide comprising at least two polypeptides fused together either directly or with the use of spacer amino acids. The fused polypeptides may serve collaborative or opposing roles in the overall function of the fusion protein.

Fusion polypeptides may further possess additional structural modifications not shared with the same organically synthesized peptide, such as adenylation, carboxylation, glycosylation, hydroxylation, methylation, phosphorylation or myristylation. These added structural modifications may be further selected or preferred by the appropriate choice of recombinant expression system. On the other hand, fusion polypeptides may have their sequence extended by the principles and practice of organic synthesis.

The present invention thus provides isolated polypeptides comprising an amino acid and/or mineral efflux transporter fused to additional polypeptides. The present invention also provides isolated polypeptides comprising an amino acid and/or mineral influx transporter fused to additional polypeptides. The additional polypeptides may be fragments of a larger polypeptide. In one embodiment, there are one, two, three, four, or more additional polypeptides fused to the influx or efflux transporter. In some embodiments, the additional polypeptides are fused toward the amino terminus of the influx or efflux transporter protein. In other embodiments, the additional polypeptides are fused toward the carboxyl terminus of the influx or efflux transporter protein. In further embodiments, the additional polypeptides flank the influx or efflux transporter protein. In some embodiments, the nucleic acid molecules encode a fusion protein comprising nucleic acids fused to the nucleic acid encoding the efflux or influx transporter. The fused nucleic acid may encode polypeptides that may aid in purification and/or immunogenicity and/or stability without shifting the codon reading frame of the influx or efflux transporter. In some embodiments, the fused nucleic acid will encode for a polypeptide to aid purification of the efflux or influx transporter. In some embodiments the fused nucleic acid will encode for an epitope and/or an affinity tag. In other embodiments, the fused nucleic acid will encode for a polypeptide that correlates to a site directed for, or prone to, cleavage. In certain embodiments, the fused nucleic acid will encode for polypeptides that are sites of enzymatic cleavage. In further embodiments, the enzymatic cleavage will aid in isolating the influx or efflux transporter protein.

The wild-type or genetically modified amino acid and/or mineral efflux transporters or amino acid and/or mineral influx transporters of the present invention may be expressed in any location in the cell, including the cytoplasm, cell surface or subcellular organelles such as the nucleus, vesicles, ER, vacuole, etc. Likewise, the additional amino acid and/or mineral influx transporters of the present invention may be expressed in any location in the cell, including the cytoplasm, cell surface or subcellular organelles such as the nucleus, vesicles, ER, vacuole, etc. Methods and vector components for targeting the expression of proteins to different cellular compartments are well known in the art, with the choice dependent on the particular cell or organism in which the transporter is expressed. See, for instance, Okumoto et al. PNAS 102: 8740-8745, 2005; Fehr et al. J. Fluoresc. 14: 603-609, 2005. Transport of protein to a subcellular compartment such as the chloroplast, vacuole, peroxisome, glyoxysome, cell wall or mitochondrion or for secretion into the apoplast, may be accomplished by means of operably linking a nucleotide sequence encoding a signal sequence to the 5′ and/or 3′ region of a gene encoding the influx or efflux transporter. Targeting sequences at the 5′ and/or 3′ end of the structural gene may determine during protein synthesis and processing where the encoded protein is ultimately compartmentalized.

The presence of a signal sequence directs a polypeptide to either an intracellular organelle or subcellular compartment or for secretion to the apoplast. The term targeting signal sequence refers to amino acid sequences, the presence of which in an expressed protein targets it to a specific subcellular localization. For example, corresponding targeting signals may lead to the secretion of the expressed amino acid and/or mineral efflux or influx transporter, e.g. from a bacterial host in order to simplify its purification. In one embodiment, targeting of the amino acid and/or mineral efflux or influx transporter may be used to affect the concentration of an amino acid and/or mineral in a specific subcellular or extracellular compartment. Appropriate targeting signal sequences useful for different groups of organisms are known to the person skilled in the art and may be retrieved from the literature or sequence data bases.

If targeting to the plastids of plant cells is desired, the following targeting signal peptides can for instance be used: amino acid residues 1 to 124 of Arabidopsis thaliana plastidial RNA polymerase (AtRpoT 3) (Plant Journal 17: 557-561, 1999); the targeting signal peptide of the plastidic Ferredoxin:NADP+ oxidoreductase (FNR) of spinach (Jansen et al. Current Genetics 13: 517-522, 1988) in particular, the amino acid sequence encoded by the nucleotides −171 to 165 of the cDNA sequence disclosed therein; the transit peptide of the waxy protein of maize including or without the first 34 amino acid residues of the mature waxy protein (Klosgen et al. Mol. Gen. Genet. 217: 155-161, 1989); the signal peptides of the ribulose bisphosphate carboxylase small subunit (Wolter et al. PNAS 85: 846-850, 1988; Nawrath et al. PNAS 91: 12760-12764, 1994), of the NADP malat dehydrogenase (Gallardo et al. Planta 197: 324-332, 1995), of the glutathione reductase (Creissen et al. Plant J. 8: 167-175, 1995) or of the R1 protein (Lorberth et al. Nature Biotechnology 16: 473-477, 1998).

Targeting to the mitochondria of plant cells may be accomplished by using the following targeting signal peptides: amino acid residues 1 to 131 of Arabidopsis thaliana mitochondrial RNA polymerase (AtRpoT 1) (Plant Journal 17: 557-561, 1999) or the transit peptide described by Braun (EMBO J. 11:3219-3227, 1992).

Targeting to the vacuole in plant cells may be achieved by using the following targeting signal peptides: The N-terminal sequence (146 amino acids) of the patatin protein (Sonnewald et al. Plant J. 1: 95-106, 1991) or the signal sequences described by Matsuoka and Neuhaus (Journal of Exp. Botany 50: 165-174, 1999); Chrispeels and Raikhel (Cell 68: 613-616, 1992); Matsuoka and Nakamura (PNAS 88: 834-838, 1991); Bednarek and Raikhel (Plant Cell 3: 1195-1206, 1991) or Nakamura and Matsuoka (Plant Phys. 101: 1-5, 1993).

Targeting to the ER in plant cells may be achieved by using, e.g., the ER targeting peptide HKTMLPLPLIPSLLLSLSSAEF in conjunction with the C-terminal extension HDEL (Haselhoff, PNAS 94: 2122-2127, 1997). Targeting to the nucleus of plant cells may be achieved by using, e.g., the nuclear localization signal (NLS) of the tobacco C2 polypeptide QPSLKRMKIQPSSQP.

Targeting to the extracellular space may be achieved by using e.g. one of the following transit peptides: the signal sequence of the proteinase inhibitor II-gene (Keil et al. Nucleic Acid Res. 14: 5641-5650, 1986; von Schaewen et al. EMBO J. 9: 30-33, 1990), of the levansucrase gene from Erwinia amylovora (Geier and Geider, Phys. Mol. Plant Pathol. 42: 387-404, 1993), of a fragment of the patatin gene B33 from Solanum tuberosum, which encodes the first 33 amino acids (Rosahl et al. Mol Gen. Genet. 203: 214-220, 1986) or of the one described by Oshima et al. (Nucleic Acids Res. 18: 181, 1990).

Additional targeting to the plasma membrane of plant cells may be achieved by fusion to a transporter, preferentially to the sucrose transporter SUT1 (Riesmeier, EMBO J. 11: 4705-4713, 1992). Targeting to different intracellular membranes may be achieved by fusion to membrane proteins present in the specific compartments such as vacuolar water channels (γTIP) (Karlsson, Plant J. 21: 83-90, 2000), MCF proteins in mitochondria (Kuan, Crit. Rev. Biochem. Mol. Biol. 28: 209-233, 1993), triosephosphate translocator in inner envelopes of plastids (Flugge, EMBO J. 8: 39-46, 1989) and photosystems in thylacoids.

Targeting to the golgi apparatus can be accomplished using the C-terminal recognition sequence K(X)KXX where “X” is any amino acid (Garabet, Methods Enzymol. 332: 77-87, 2001

Targeting to the peroxisomes can be done using the peroxisomal targeting sequence PTS I or PTS II (Garabet, Methods Enzymol. 332: 77-87, 2001).

Methods for the introduction of nucleic acid molecules into plants are well-known in the art. For example, plant transformation may be carried out using Agrobacterium-mediated gene transfer, microinjection, electroporation or biolistic methods as it is, e.g., described in Potrykus and Spangenberg (Eds.), Gene Transfer to Plants. Springer Verlag, Berlin, N.Y., 1995. Therein, and in numerous other references available to one of skill in the art, useful plant transformation vectors, selection methods for transformed cells and tissue as well as regeneration techniques are described and can be applied to the methods of the present invention.

Accordingly, the present invention also relates to methods of producing pathogen-resistant plant cells. In one embodiment, the methods comprise identifying at least one amino acid efflux transporter and/or at least one mineral efflux transporter wherein the levels of expression or activity of the at least one amino acid efflux transporter and/or at least one mineral efflux transporter are increased in the plant cell in response to an infection of the pathogen as compared to an uninfected plant cell. Subsequently, the plant cell is modified to inhibit the activity or reduce the expression of the at least one identified amino acid efflux and/or the at least one mineral efflux transporter, where inhibiting the activity or reducing the expression of the at least one identified amino acid efflux transporter and/or the at least one efflux mineral efflux produces the pathogen-resistant plant cell.

In another embodiment, the methods comprise identifying at least one amino acid influx transporter and/or at least one mineral influx transporter wherein the levels of expression or activity of at least one amino acid influx transporter and/or at least one mineral influx transporter are increased in the plant cell in response to an infection with the pathogen as compared to an uninfected plant cell. Subsequently, the plant cell is modified to inhibit the activity or reduce expression of the at least one identified amino acid influx transporter and/or at least one identified mineral influx transporter, where inhibiting the activity or reducing the expression of the at least one identified amino acid influx transporter and/or at least one mineral influx transporter produces the pathogen-resistant plant cell.

In another embodiment, the methods comprise identifying at least one amino acid efflux transporter and/or at least one mineral efflux transporter wherein the levels of expression or activity of the at least one amino acid efflux transporter and/or at least one mineral efflux transporter are decreased in the plant cell in response to an infection of the pathogen as compared to an uninfected plant cell. Subsequently, the plant cell is modified to increase the activity or the expression of the at least one identified amino acid efflux and/or the at least one mineral efflux transporter, where increasing the activity or the expression of the at least one identified amino acid efflux transporter and/or the at least one efflux mineral efflux produces the pathogen-resistant plant cell.

In another embodiment, the methods comprise identifying at least one amino acid influx transporter and/or at least one mineral influx transporter wherein the levels of expression or activity of at least one amino acid influx transporter and/or at least one mineral influx transporter are decreased in the plant cell in response to an infection with the pathogen as compared to an uninfected plant cell. Subsequently, the plant cell is modified to increase the activity or expression of the at least one identified amino acid influx transporter and/or at least one identified mineral influx transporter, where increasing the activity or the expression of the at least one identified amino acid influx transporter and/or at least one mineral influx transporter produces the pathogen-resistant plant cell.

Methods of identifying transporters whose expression is decreased or increased in response to exposure to a pathogen are well known in the art. For example, in one embodiment, plant cells are co-cultured with a pathogen and an expression array is performed on RNA isolated from the plant cells. The expression array can identify which genes are upregulated and down regulated in response to the pathogen. Of course, different plant cells and different pathogens can be combined in various assays to identify the appropriate efflux and influx transporters. For example, Wang, Y. et al. MPMIm 18(5):385-396 (2005) discloses microarray analysis of gene expression profiles in response to inoculating plant cells with Rhizobacteria.

In another aspect, the invention provides harvestable parts or plants and methods to propagate material of the transgenic plants according to the invention which contain transgenic plant cells as described above. Harvestable parts can be in principle any useful part of a plant, for example, leaves, stems, fruit, seeds, roots etc. Propagation material includes, for example, seeds, fruits, cuttings, seedlings, tubers, rootstocks etc.

The examples herein are illustrative in nature and are not intended to limit the scope of the invention.

### EXAMPLES

### Example 1

Most studies of the plant response to either pathogens or microbe-associated molecular patterns (MAMPs) have been carried out using either mature plants or plant tissue culture cells. To provide an alternative system to facilitate the study of defense signaling pathways, an Arabidopsis thaliana model was developed that utilizes ten-day old Arabidopsis seedlings treated with MAMPs including the synthetic flagellin peptide Flg22 or that utilizes ten-day old Arabidopsis seedlings infected with bacterial pathogens in multi-well plates. Using this system studies were performed to determine the mechanisms by which seedlings can be elicited to become resistant to bacterial infection. See Clay, N. K., et al., Science, 323:95-101 (2009); Danna, C. H., et al., Proc Natl Acad Sci USA, 108:9286-9291 (2011); Denoux, C., R. et al., Characterization of Arabidopsis MAMP response pathways, In M. Lorito, et al., (eds.), Biology of Plant-Microbe Interactions, vol. 6. International Society of Molecular Plant-Microbe Interactions (1998); Denoux, C., R. et al., Mol Plant, 1:423-445 (2008); Millet, Y. A., et al., Plant Cell, 22:973-990 (2010); Songnuan, W., et al., A seedling assay for MAMP signaling and infection studies, In M. Lorito, et al., (eds.), Biology of Plant-Microbe Interactions, vol. 6. International Society for Molecular Plant-Microbe Interactions (2008), all of which are incorporated by reference.

The seedlings were germinated and grown in liquid medium under sterile conditions. In a typical assay, each well of a 12-well plate contained 15-20 seedlings in 1 ml of filter-sterilized Murashige and Skoog Basal medium supplemented with 0.5% sucrose, pH 5.7. This setup provides enough replicates to average out biological variations. For high-throughput assays, seedlings can be grown in 96-well plates and used in genetic or chemical screens. Chemicals, hormones, elicitors, or pathogens were added directly into the medium. The plates were wrapped with parafilm to prevent evaporation and placed at 22° C. under a 16 hours light/8 hours dark photoperiod with a light intensity of 100 μEm⁻²s⁻¹. After 8 days, the medium was replaced with a fresh batch to replenish the nutrients and equalize the volume of liquid in the wells. On day 10, seedlings were treated by adding desired concentration of MAMPs, supplements, or bacteria directly into the liquid medium.

Pseudomonas syringae pv. tomato strain DC3000 (Pst DC3000) or P. syringae pv. maculicola strain ES4326 (Psm ES4326) grow rapidly until about 24 hours after inoculation. For Pseudomonas syringae infection, bacteria were harvested in log phase, thoroughly rinsed with the plant medium, resuspended to OD600=0.002 in water. 100 μl of the suspension was added to each well of a 12-well plate.

For assays in 12-well plates, bacterial inoculation was carried out by adding suspended bacterial cells directly into the media. Importantly, none of these bacterial strains can grow repidly in the MS liquid medium if the seedlings are not present, even though the medium contains plentiful amounts of sugar and inorganic sources of nitrogen. Symptoms were monitored for several days after inoculation. The number of bacteria inside the seedlings was quantified in the seedling infection assays. Seedlings were blotted dry on absorbent paper, transferred to a round-bottom 2 mL eppendorf tube containing 400 ul of water and ground with a Tissue Lyser (QIAGEN). 100 μl of seedlings lysate were transferred to a solid bottom white 96-well plate and bacterial CFUs were assessed by measuring luciferase activity.

To determine whether the recognition of MAMP elicitors by seedlings resulted in a biologically significant response, it was tested whether the flagellin-derived synthetic peptide Flg22 triggered enhanced resistance in seedlings to P. syringae. By using Psm ES4326 or Pst DC3000 strains expressing the LUX operon from Photorhabdus luminescens, it was possible to monitor bacterial growth by measuring light emission in a scintillation counter. Seedlings were grown in medium for 10 days and subsequently treated with Flg22 for 24 hours before inoculation with Psm-LUX. To assess bacterial growth inside seedlings, 400 ul of water were added and seedlings were ground in a round-bottom 2 mL eppendorf tube. 100 ul of seedlings lysate were transferred to a solid bottom white 96-well plate for LUX activity detection. Bacterial growth was estimated by converting light emission into CFUs (using an experimentally-determined CPMs/CFUs conversion table).

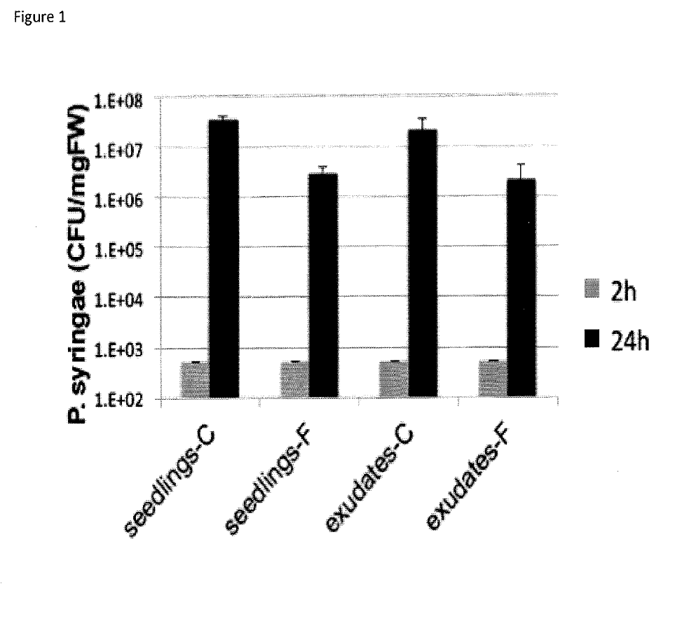

As shown in FIG. 1, Flg22 elicited protection against Pst-LUX growth in seedlings. Moreover, the medium (exudate) in which seedlings were grown supported significantly more growth than medium from plants elicited by Flg22. These results support the hypothesis that Flg22 treatment decreases the amount of nutrients that are required to support the growth of the bacteria by either suppressing efflux or activating influx of nutrients.

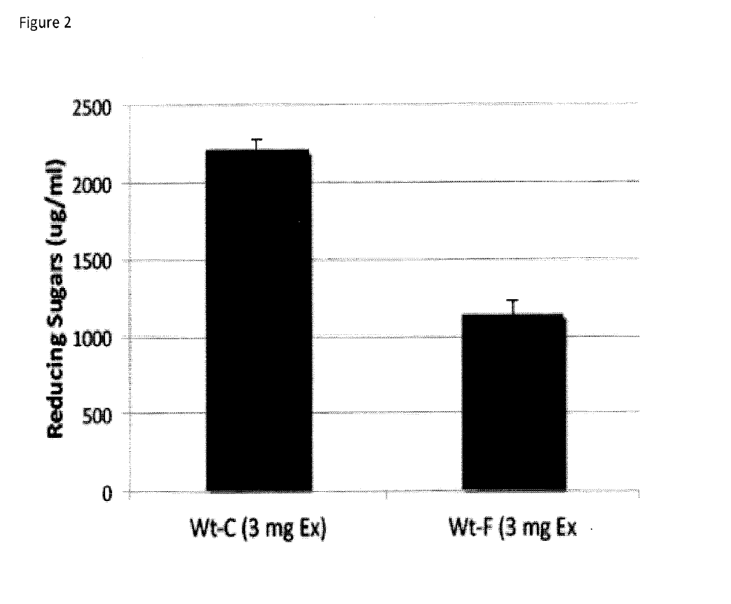

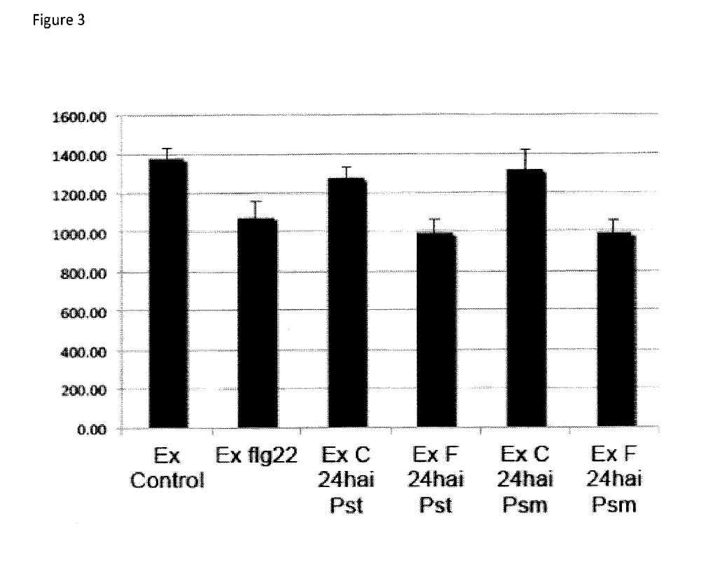

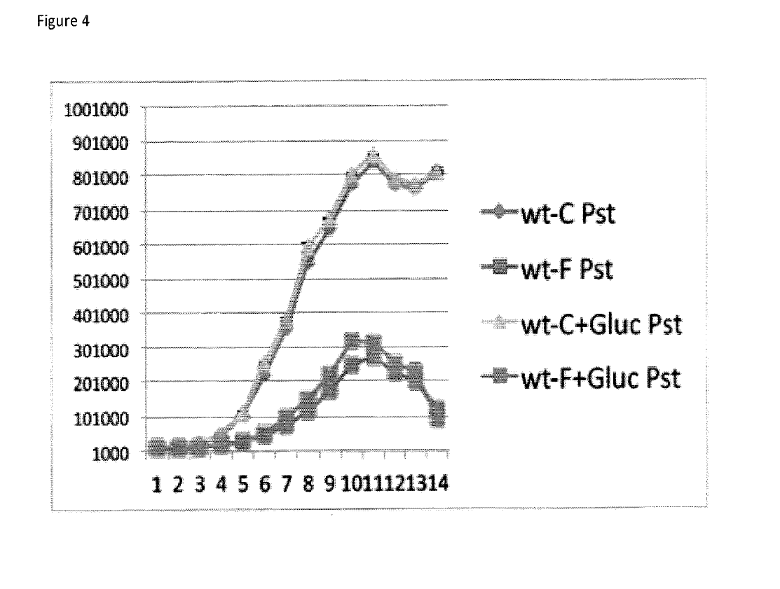

To confirm that Flg22 treatment decreases the amount of nutrients in the medium, levels of sugars were measured in medium in which seedlings had grown with and without Flg22 treatment. As shown in FIG. 2, Flg22 treatment resulted in an approximate 50% reduction in the levels of reducing sugars in the exudates, providing support for the nutrient deprivation model. On the other hand, overnight growth of P. syringae in the exudate medium had only a marginal effect on the levels of reducing sugars in the exudates suggesting that sugars are not limiting bacterial growth (FIG. 3). Moreover, supplementation of the seedling infection assay with glucose had no discernable effect on the growth of P. syringae in the seedlings (FIG. 4). These latter experiments (FIGS. 3 and 4) show that if nutrient deprivation is a key factor in restricting the growth of P. syringae after Flg22 treatment, but sugars are not the limiting, or at least not the only limiting nutrients.

### Example 2

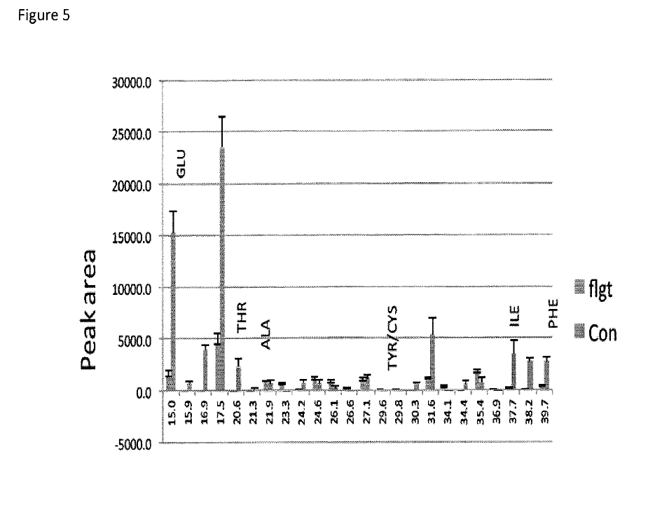

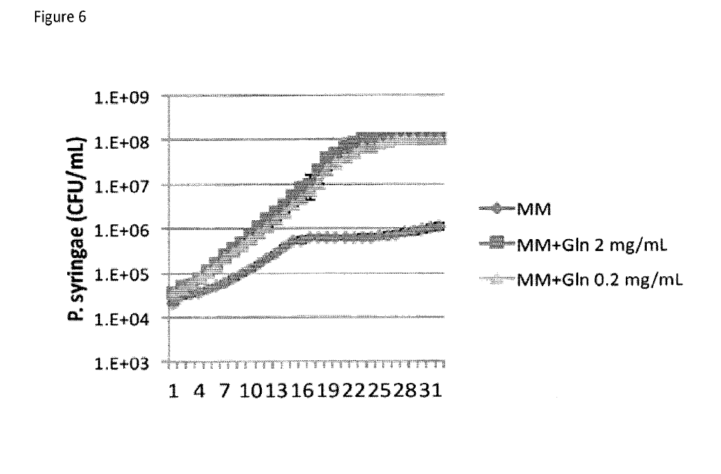

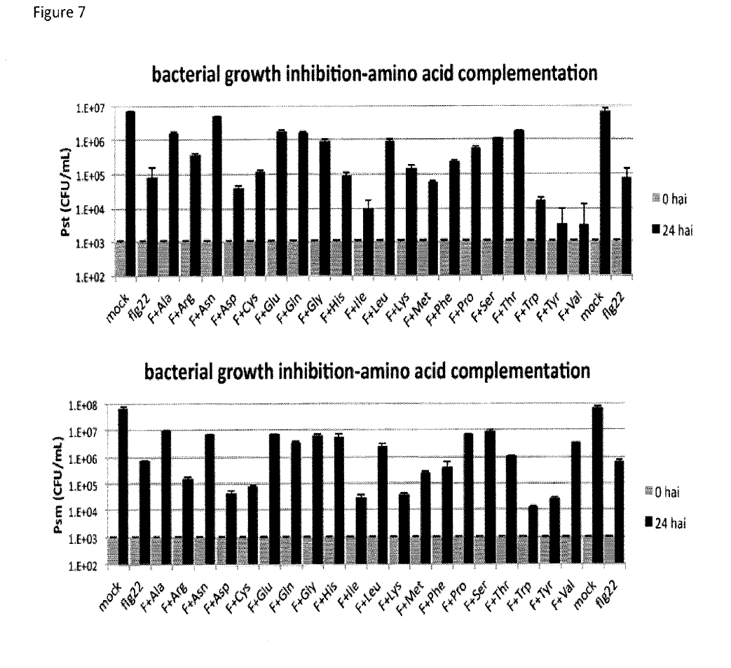

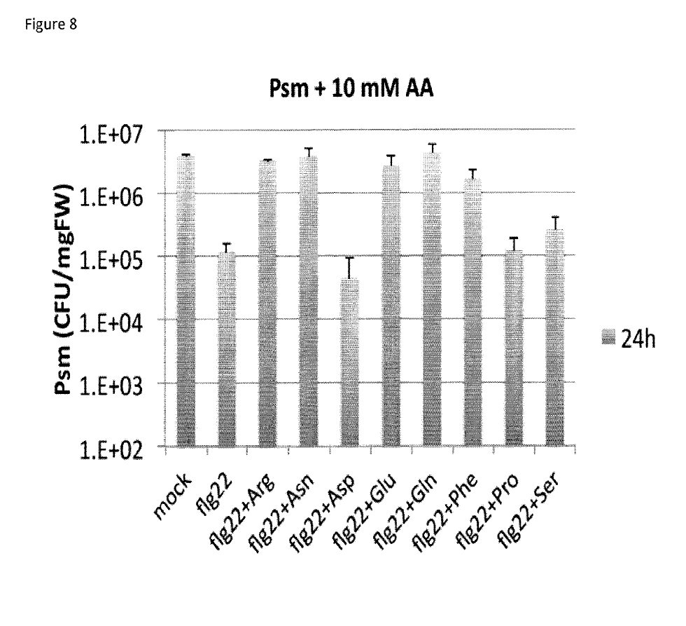

In most environments, the nutrient that is most limiting to pathogen survival in plants is nitrogen, not carbon. To test whether Flg22 treatment of seedlings affected the levels of nitrogen-containing amino acids in the exudates, HPLC analysis using individual amino acids as standards was performed. As shown in FIG. 5, Flg22 treatment resulted in a significant decrease in the levels of several amino acids in the exudates, gluthamate among others. The addition of glutamate to flg22-treated seedlings, allowed Pst to grow (FIG. 6) to almost the same extent as it does on control mock treated seedlings. In addition, several amino acids suppressed the restriction of bacterial growth elicited by Flg22 in both exduates (FIG. 7) and in seedlings (FIG. 8). The data in FIGS. 5-8 demonstrate that Flg22 treatment of seedlings resulted in either the sequestering or uptake of amino acids or the suppression of amino acid secretion, or both mechanisms at the same time, which directly resulted in restricting the growth of P. syringae both in the exudate medium and in the seedlings. Preliminary data indicate that the same nutrient withdrawal mechanisms operate in adult plants growing on soil. Infiltration of Arabidopsis leaves with flg22 prior to bacterial inoculation strongly reduces bacterial growth compared to mock pre-infiltrated leaves (Zipfel et al. Nature 2004, which is incorporated by reference). The co-infiltration of bacteria with amino acids, allows bacteria to growth on flg22 treated leaves to levels coparable to those of mock treated leaves, which suggest that amino acids are being withdrawn from the apoplast of adult plants after flg22 perception, which in turns is suppressing bacterial growth.

### Example 3

Flg22 treatment resulted in the up regulation of genes that encode transporters involved in the uptake of amino acids and sugars. For example, MATE transporters, which are involved in the efflux of compounds from cells, such as MtN21, (as well as other potential transporters) are involved in conferring resistance to pathogens by a nutrient withdrawal mechanism. A review of published genes identified several known glucose transporters and several putative amino acid transporters that are up or down regulated transcriptionally after Flg22 perception. In several specific embodiments, the genes listed in Table 1 below can be used in the methods and plant cells of the present invention.

Among amino acid transporter-encoding genes, three families were identified with members showing transcriptional up or down regulation after Flg22 elicitation: (1) genes encoding amino acid transporters belonging to the Medicago truncatula nodulin 21 (Eama-like transporters)(“MtN21”), (2) genes encoding amino acid permease transporters, and (3) and genes encoding cationic amino acid transporters.

Genes encoding cationic amino acid transporters (AAT1/CAT1, CAT5 and AT1G80510), which have been shown to participate in the re-uptake of His, Arg and Glu from the vasculature and intercellular space (Su, Y. H., et al., Plant Physiol., 136:3104-3113 (2004), which is incorporated by reference), were strongly up-regulated in response to Flg22, demonstrating that active mechanisms leading to amino acid withdrawal from the intercellular space is being turned on after Flg22 perception. Members of the amino acid permease family (AAP4, AAP3, LHT1, LHT7 and ProT2) (Lalonde, S., et al., Annu Rev Plant Biol, 55:341-372 (2004), which is incorporated by reference), which are involved in re-uptaking amino acids from the vascular system and the intercellular space of mesophyll cells into the cytoplasm (Hirner, A., et al. Plant Cell, 18:1931-1946 (2006), which is incorporated by reference), were also strongly up-regulated after Flg22 elicitation. Finally, a family of genes with at least one member known to play a positive role in regulating the loading of amino acid from the cytosol into the intercellular space (See Pilot, G., et al., Plant Cell, 16:1827-1840 (2004); Pratelli, R., et al., The ubiquitin E3 ligase LOSS OF GDU 2 is required for GLUTAMINE DUMPER 1-induced amino secretion in Arabidopsis. Plant physiology, (2012); and Pratelli, R., et al., Plant Physiology, 152:762-773 (2012), all of which are incorporated by reference), showed strong down-regulation after Flg22 elicitation (GDU4 and GDU7).

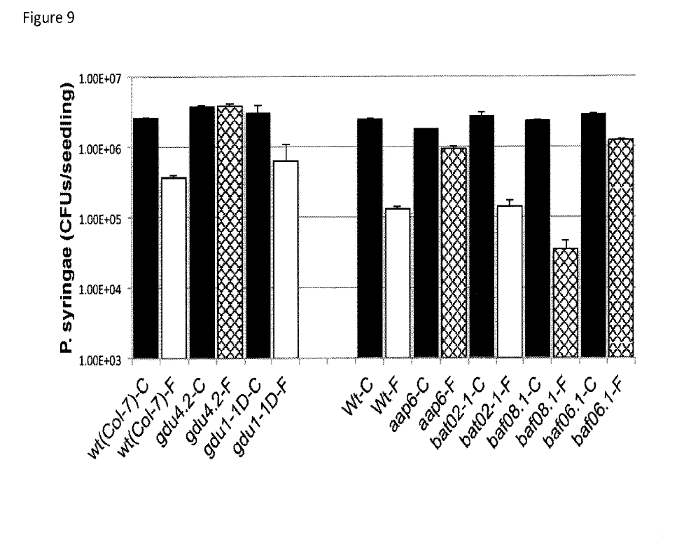

Among the genes belonging to the NtN21 family of transporters, BAF08 was transcriptionally down regulated upon flg22-treatment. The down-regulation of BAF08 suggests that this transporter could function as an exporter of amino acids, which needs to be down-regulated to promote the withdrawal of amino acids from the apoplast upon flg22 elicitation. This proposed function for BAF08 is consistent with the hyperresponsive phenotype that was observed in a loss of function mutant in which BAF08 was inactivated by a T-DNA insertion (See FIG. 9). That is, in a BAF08 loss of function mutant, one might expect to find lower amino acid levels in the apoplast following flg22 treatment compared to a wild type plant. A second member of the MtN21 family, BAF06, was also transcriptionally down regulated by flg22 but a loss of function mutant of this gene was compromised for bacterial growth inhibition after elicitation with flg22 (FIG. 9), suggesting that the BAF06 protein could localize in cells whose function is to load the vasculature with amino acids. Thus, the inactivation of BAF06 could cause an over-accumulation of amino acids in the apoplast of mesophyll cells where bacteria propagate. A third member of the MtN21 family baf02, which was mildly induced by flg22 (2.1 fold) and did not seem to be essential for the withdrawal of amino acids as the loss of function mutant still showed a bacterial growth inhibition response comparable to the one observed in wild type plants (FIG. 9).

A loss of function mutant for one of the members of the amino acid permease family, AAP6, which was transcriptionally induced after flagellin elicitation, was compromised for bacterial growth inhibition after flg22 treatment (FIG. 9). In line with the withdrawal hypothesis triggered by flg22, AAP6 likely functions as an importer of amino acid into the vascular system. Mutations in AAP6 have been shown to cause a reduced load of amino acids into the vasculature, suggesting that elicitation of seedlings with flg22 could trigger the withdrawal of amino acids in the apoplast by activating the loading of the vascular system.

Among the gene family whose members are proposed to have a regulatory function on amino acid transport, GDU4 and GDU7 were strongly down-regulated by flg22 treatment. A gain-of-function mutant in GDU4 (gdu4.2) was compromised for inhibiting bacterial growth after flg22 treatment, suggesting that GDU4 functions as a positive regulator of amino acid secretion into the apoplast, a function that needs to be suppressed by flg22 to execute the withdrawal response and the subsequent inhibition of bacterial growth.

MATE transporters are involved in efflux of compounds from cells. For example ALMT exports malate (Ligaba, A., et al., Maize ZmALMT2 is a root anion transporter that mediates constitutive root malate efflux. Plant Cell Environ. (2011), which is incorporated by reference), and mammalian MATEs export a variety of compounds including xenobiotics. Other MATEs have been implied in pathogen resistance, e.g., ADS1 encodes a MATE-transporter that negatively regulates plant disease resistance (Sun, X., et al., The New Phytologist, 192:471-482 (2011), which is incorporated by reference).

TABLE 1
Candidate Genes - Current List

| Gene ID | Gene Name | Fcn. Of Gene/Identity |
|---|---|---|
| At1g14880 | PCR1 | Cadmium transport |
| At1g14870 | PCR2 | Zinc transport |
| At1g52200 | PCR8 | Cadmium/Zinc related |
| | | Transport |
| At1g05300 | Zip5 | Zinc transport |
| At5g53550 | YSL 3 | Chelated metal transporter |
| | | in Plasma membrane. |
| At4g01430 | Mtn21-family Nodulin- | Mtn21-family Nodulin- |
| | Related ene | Related Gene |
| At2g39210 | Mtn21-family Nodulin- | Mtn21-family Nodulin- |
| | Related Gene | Related Gene |
| At3g56620 | Mtn21-family Nodulin- | Mtn21-family Nodulin- |
| | Related Gene | Related Gene |
| At3g53210 | Mtn21-family Nodulin- | Mtn21-family Nodulin- |
| | Related Gene | Related Gene |
| At1g57980/990 | Pup18/Pup | Purine Transporter |
| At4g21680 | NRT1.8 | Nitrate transporter in |
| | | nitrate removal from xylem |
| | | sap |
| At1g44800 | Mtn21-family | Mtn21-family Nodulin- |
| | | Related Gene |
| At2g37460 | Mtn21-family | Mtn21-family Nodulin- |
| | | Related Gene |
| At5g40210 | Mtn21 | Mtn21-family Nodulin- |
| | | Related Gene |
| At2g16660 | Nodulin like (but not | Substrate transporter |
| | MtN21 fam.) | |

One of skill in the art that the specific genes disclosed herein include homologs to these same genes in other organisms. For example, the plant cells and methods of the present invention encompass reducing the expression or activity of homologs of the At1g44800 gene in Arabidopsis.

The following References are incorporated by references in their entirety:
1. Chen, L. Q., et al., Nature, 468:527-532 (2010).
2. Chen, L. Q., et al., Science, 335:207-211 (2012).
3. Clay, N. K., et al., Science, 323:95-101 (2009).
4. Danna, C. H., et al., Proc Natl Acad Sci USA, 108:9286-9291 (2011).
5. Denoux, C., et al., Characterization of Arabidopsis MAMP response pathways, In M. Lorito, et al., (eds.), Biology of Plant-Microbe Interactions, vol. 6. International Society of Molecular Plant-Microbe Interactions, St. Paul, Minn. (1988).
6. Denoux, C., et al., Mol Plant, 1:423-445 (2008).
7. Heller, G., et al. Expression analysis of Clavata1-like and Nodulin21-like genes from Pinus sylvestris during ectomycorrhiza formation. Mycorrhiza (2011).
8. Hirner, A., et al., Plant Cell, 18:1931-1946 (2006).
9. Hirner, A., et al, Plant Cell, 18:1931-1946 (2006).
10. Hvorup, R. N., et al., Eur J Biochem, 270:799-813 (2003).
11. Lalonde, S., et al., Annu Rev Plant Biol, 55:341-372 (2004).
12. Ligaba, A., et al., Maize ZmALMT2 is a root anion transporter that mediates constitutive root malate efflux. Plant Cell Environ, (2011).
13. Matsye, P. D., et al., Plant Mol Biol, 77:513-528 (2011).
14. Millet, Y. A., et al., Plant Cell, 22:973-990 (2010).
15. Nawrath, C., et al., Plant Cell, 14:275-286 (2002).
16. Pilot, G., et al., Plant Cell, 16:1827-1840 (2004).
17. Pratelli, R., et al., The ubiquitin E3 ligase LOSS OF GDU 2 is required for GLUTAMINE DUMPER 1-induced amino secretion in Arabidopsis. Plant physiology (2012).
18. Pratelli, R., et al., Plant Physiology, 152:762-773 (2010).
19. Sauer, N., and J. Stolz. Plant J, 6:67-77 (1994).
20. Songnuan, W., et al., A seedling assay for MAMP signaling and infection studies, In M. Lorito, et al., (eds.), Biology of Plant-Microbe Interactions, vol. 6. International Society for Molecular Plant-Microbe Interactions, St. Paul, Minn. (2008).
21. Su, Y. H., et al., Plant Physiol, 136:3104-3113 (2004).
22. Sun, X., et al., The New Phytologist, 192:471-482 (2011).
23. Zhao, Y., et al., Identification and Fine Mapping of rhm1 Locus for Resistance to Southern Corn Leaf Blight in Maize. J Integr Plant Biol. (2012).

1. A genetically modified plant cell that has altered expression or activity of at least one amino acid efflux transporter compared to levels of expression or activity of the at least one amino acid efflux transporter in an unmodified plant cell.

2. The genetically modified plant cell of claim 1, wherein the at least one amino acid efflux transporter is a member of a family of proteins selected from the group consisting of the MtN21 family, the amino acid permease transporter family and the cationic amino acid transporter family.

3. The genetically modified plant cell of claim 2, wherein the at least one amino acid efflux transporter is selected from the group consisting of to a cysteine transporter, a histidine transporter, an isoleucine transporter, a methionine transporter, a serine transporter, a valine transporter, an alanine transporter, a glycine transporter, a leucine transporter, a proline transporter, a threonine transporter, a phenylalanine transporter, an arginine transporter, a tyrosine transporter, a tryptophan transporter, an aspartate transporter, an asparagine transporter, a glutamate transporter, a glutamine transporter, a lysine transporter and any combination thereof.

4. The genetically modified plant cell of claim 2, wherein the at least one amino acid efflux transporter is encoded by a gene selected from the group consisting of AAT/CAT1, CAT5, AT1G80510, AAP4, AAP3, LHT7, ProT2, GDU4 and GDU7.

5-14. (canceled)

15. A method of producing a pathogen-resistant plant cell, the method comprising
a) identifying at least one amino acid efflux transporter wherein the levels of expression or activity of the at least one amino acid efflux transporter are altered in the plant cell in response to an infection of the pathogen as compared to an uninfected plant cell, and
b) genetically modifying the plant cell to either (i) inhibit the activity or reduce the expression of the at least one identified amino acid efflux transporter in (a), or (ii) increase the activity or expression of the at least one identified amino acid efflux transporter in (a),
whereby inhibiting the activity or reducing the expression of the at least one identified amino acid efflux transporter or whereby increasing the activity or the expression of the at least one identified amino acid efflux transporter produces the pathogen-resistant plant cell.

16. The method of claim 15, wherein the at least one amino acid efflux transporter is a member of a family of proteins selected from the group consisting of the MtN21 family, the amino acid permease transporter family and the cationic amino acid transporter family.

17. The method of claim 16, wherein the at least one amino acid efflux transporter is selected from the group consisting of a cysteine transporter, a histidine transporter, an isoleucine transporter, a methionine transporter, a serine transporter, a valine transporter, an alanine transporter, a glycine transporter, a leucine transporter, a proline transporter, a threonine transporter, a phenylalanine transporter, an arginine transporter, a tyrosine transporter, a tryptophan transporter, an aspartate transporter, an asparagine transporter, a glutamate transporter, a glutamine transporter, a lysine transporter and any combination thereof.

18-27. (canceled)

28. A genetically modified plant cell that has altered expression or activity of at least one mineral efflux transporter compared to levels of expression or activity of the at least one mineral efflux transporter in an unmodified plant cell.

29. The genetically modified plant cell of claim 28, wherein the at least one mineral efflux transporter is selected from the group consisting of a zinc transporter, a cadmium transporter, and an iron transporter.

30. The genetically modified plant cell of claim 29, wherein the at least one mineral efflux transporter is encoded by a gene selected from the group consisting of AT1G05300 and AT5G53550.

31-40. (canceled)

41. A method of producing a pathogen-resistant plant cell, the method comprising
a) identifying at least one mineral efflux transporter wherein the levels of expression or activity of the at least one mineral efflux transporter are altered in the plant cell in response to an infection of the pathogen as compared to an uninfected plant cell, and
b) genetically modifying the plant cell to either (i) inhibit the activity or reduce the expression of the at least one identified mineral efflux transporter in (a), or (ii) increase the activity or expression of the at least one identified mineral efflux transporter in (a),
whereby inhibiting the activity or reducing the expression of the at least one identified mineral efflux transporter or whereby increasing the activity or the expression of the at least one identified mineral efflux transporter produces the pathogen-resistant plant cell.

42. The method of claim 41, wherein the at least one mineral efflux transporter is selected from the group consisting of a zinc transporter, a cadmium transporter, and an iron transporter.

43-52. (canceled)

53. A genetically modified plant cell that has altered expression or activity of at least one amino acid influx transporter compared to levels of expression or activity of the at least one amino acid influx transporter in an unmodified plant cell.

54. The genetically modified plant cell of claim 53, wherein the at least one amino acid influx transporter is selected from the group consisting of a cysteine transporter, a histidine transporter, an isoleucine transporter, a methionine transporter, a serine transporter, a valine transporter, an alanine transporter, a glycine transporter, a leucine transporter, a proline transporter, a threonine transporter, a phenylalanine transporter, an arginine transporter, a tyrosine transporter, a tryptophan transporter, an aspartate transporter, an asparagine transporter, a glutamate transporter, a glutamine transporter, a lysine transporter and any combination thereof.

55-59. (canceled)

60. A method of producing a pathogen-resistant plant cell, the method comprising
a) identifying at least one amino acid influx transporter wherein the levels of expression or activity of the at least one amino acid influx transporter are altered in the plant cell in response to an infection of the pathogen as compared to an uninfected plant cell, and
b) genetically modifying the plant cell to either (i) inhibit the activity or reduce the expression of the at least one identified amino acid influx transporter in (a), or (ii) increase the activity or expression of the at least one identified amino acid influx transporter in (a),
whereby inhibiting the activity or reducing the expression of the at least one identified amino acid influx transporter or whereby increasing the activity or the expression of the at least one identified amino acid influx transporter produces the pathogen-resistant plant cell.

61. The method of claim 60, wherein the at least one amino acid influx transporter is selected from the group consisting of a cysteine transporter, a histidine transporter, an isoleucine transporter, a methionine transporter, a serine transporter, a valine transporter, an alanine transporter, a glycine transporter, a leucine transporter, a proline transporter, a threonine transporter, a phenylalanine transporter, an arginine transporter, a tyrosine transporter, a tryptophan transporter, an aspartate transporter, an asparagine transporter, a glutamate transporter, a glutamine transporter, a lysine transporter and any combination thereof.

62-65. (canceled)

66. A genetically modified plant cell that has altered expression or activity of at least one mineral influx transporter compared to levels of expression or activity of the at least one mineral influx transporter in an unmodified plant cell.

67. The genetically modified plant cell of claim 66, wherein the at least one mineral influx transporter is selected from the group consisting of a zinc transporter, a cadmium transporter, and an iron transporter.

68-72. (canceled)

73. A method of producing a pathogen-resistant plant cell, the method comprising
a) identifying at least one mineral influx transporter wherein the levels of expression or activity of the at least one mineral influx transporter are altered in the plant cell in response to an infection of the pathogen as compared to an uninfected plant cell, and
b) genetically modifying the plant cell to either (i) inhibit the activity or reduce the expression of the at least one identified mineral influx transporter in (a), or (ii) increase the activity or expression of the at least one identified mineral influx transporter in (a),
whereby inhibiting the activity or reducing the expression of the at least one identified mineral influx transporter or whereby increasing the activity or the expression of the at least one identified mineral influx transporter produces the pathogen-resistant plant cell.

74. The method of claim 73, wherein the at least one mineral influx transporter is selected from the group consisting of zinc transporter, a cadmium transporter, and an iron transporter.

75-78. (canceled)


SEQUENCE LISTING (4 sequences)

SEQ ID NO: 1 - protein, 22 aa, Artificial Sequence
  ER targeting peptide
HKTMLPLPLIPSLLLSLSSAEF

SEQ ID NO: 2 - protein, 15 aa, Nicotiana tabacum
QPSLKRMKIQPSSQP

SEQ ID NO: 3 - protein, 4 aa, Artificial Sequence
  C-terminal extension for targeting to the ER
HDEL

SEQ ID NO: 4 - protein, 5 aa, Artificial Sequence
  C-terminal recognition sequence for targeting to golgi apparatus
KXKXX